(19) United States
(12) Patent Application Publication
(10) Pub. No.: US 2026/0130692 A1
(43) Pub. Date: May 14, 2026

(54) SPLIT CANNULAS FOR MULTI-PORTAL SURGICAL PROCEDURES, AND ASSOCIATED SYSTEMS AND METHODS

(71) Applicant: Amplify Surgical, Inc., Irvine, CA (US)

(72) Inventors: Andy Wonyong Choi, Irvine, CA (US); Clark Hutton, Carlsbad, CA (US); Shigeru Tanaka, Half Moon Bay, CA (US); Don Park, Irvine, CA (US)

(21) Appl. No.: 19/384,720

(22) Filed: Nov. 10, 2025

Related U.S. Application Data
(60) Provisional application No. 63/719,061, filed on Nov. 11, 2024.
(60) Provisional application No. 63/736,526, filed on Dec. 19, 2024.
(60) Provisional application No. 63/779,782, filed on Mar. 28, 2025.

(51) Int. Cl. A61B 17/34; A61B 1/06

(52) U.S. Cl. CPC A61B 17/3423; A61B 1/0669; A61B 2017/347; A61B 2017/3484

(57) ABSTRACT

Systems, devices, and methods for split cannula assemblies are disclosed herein. In some embodiments, a split cannula assembly includes a split cannula and a retention device. The split cannula can include one or more openings, and the retention device can include one or more retention features and can be movable relative to the split cannula between an anchoring position for anchoring the split cannula to a patient and a non-anchoring position for moving the split cannula relative to the patient. The one or more retention features can extend through corresponding ones of the one or more openings to engage tissue of the patient when the retention device is in the anchoring position. The one or more retention features can move away from the tissue when the retention device is moved toward the non-anchoring position.

### CROSS-REFERENCE TO RELATED APPLICATIONS

The present application claims the benefit of U.S. Provisional Patent Application No. 63/719,061, filed Nov. 11, 2024, U.S. Provisional Patent Application No. 63/736,526, filed Dec. 19, 2024, and U.S. Provisional Patent Application No. 63/779,782, filed Mar. 28, 2025, the disclosures of which are incorporated herein by reference in their entireties.

### TECHNICAL FIELD

The present technology relates generally to medical systems and, more particularly, to systems, devices, and methods for performing multi-portal surgical procedures.

### BACKGROUND

Individuals often suffer from damaged or displaced spinal discs and/or vertebral bodies due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An implant (commonly referred to as an interbody spacer) can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is packed with bone and/or bone growth-inducing materials. Unfortunately, it may be difficult to implant the interbody spacer at the intended implantation site between vertebral bodies. Additionally, conventional surgical techniques can cause a significant amount of trauma at or near the implantation site (e.g., injury to nerve tissue), which can significantly increase recovery time and lead to patient discomfort.

Spinal nerve compression can be caused by narrowing of the spinal canal associated with arthritis (e.g., osteoarthritis) of the spine, degeneration of spinal discs, and thickening of ligaments. Arthritis of the spine often leads to the formation of bone spurs, which can narrow the spinal canal and press on the spinal cord. In spinal disc degeneration, inner tissue of the disc can protrude through a weakened fibrous outer covering of the disc and can press on the spinal cord and/or spinal nerve roots. Ligaments located along the spine can thicken over time and press on the spinal cord and/or nerve roots. Unfortunately, spinal nerve compression can cause lower back pain, hip pain, and/or leg pain and may also result in numbness, depending on the location of the compressed nerve tissue. For example, spinal stenosis that causes spinal cord compression in the lower back can cause numbness of the legs. It is difficult to visualize internal tissue when removing tissue, often resulting in injury or removal of nerve tissue. Accordingly, there is a need for improved surgical systems, visualization techniques, and/or related technologies.

### BRIEF DESCRIPTION OF THE DRAWINGS

Features, aspects, and advantages of the presently disclosed technology may be better understood with regard to the following drawings.

FIG. 1 is a side view of a multi-portal spinal surgical system configured in accordance with embodiments of the present technology.

FIGS. 2A and 2B are schematic top plan and isometric views, respectively, showing surgical approaches to a lumbar spine for performing procedures.

FIGS. 3-5B illustrate surgical steps for performing spinal procedures in accordance with embodiments of the present technology.

FIGS. 6A-6E are front isometric, rear isometric, side, top, and enlarged side views, respectively, of a split cannula assembly configured in accordance with embodiments of the present technology.

FIG. 7 is a detailed view of a motion inhibitor of the split cannula assembly of FIG. 6C, configured in accordance with embodiments of the present technology.

FIGS. 7A and 7B are front partially exploded and rear partially exploded views, respectively, of the split cannula assembly of FIG. 6A.

FIGS. 8A-8D are front isometric, rear isometric, side, and top views, respectively, of a split cannula assembly configured in accordance with embodiments of the present technology.

FIGS. 9A and 9B are front partially exploded and rear partially exploded views, respectively, of the split cannula assembly of FIG. 8A.

FIGS. 10A-10D are front isometric, rear isometric, side, and top views, respectively, of a split cannula assembly configured in accordance with embodiments of the present technology.

FIGS. 11A and 11B are front partially exploded and rear partially exploded views, respectively, of the split cannula assembly of FIG. 10A.

FIGS. 12A-12D are front isometric, rear isometric, side, and top views, respectively, of a split cannula assembly configured in accordance with embodiments of the present technology.

FIGS. 13A and 13B are front partially exploded and rear partially exploded views, respectively, of the split cannula assembly of FIG. 12A.

FIG. 14 is a side view of an expander of the split cannula assembly of FIG. 12A.

FIG. 14A is a cross-sectional view of the expander taken along line 14A-14A of FIG. 14.

FIG. 15 is a side cross-sectional view of the expander taken along line 15-15 of FIG. 13B.

FIGS. 16A and 16B are front and rear views, respectively, of the split cannula assembly of FIG. 12A in an expanded state.

FIGS. 17 and 18 illustrate expansion of the split cannula assembly of FIG. 12A in accordance with embodiments of the present technology.

FIGS. 19A-19C are isometric, side, and front views, respectively, of a cannula in accordance with embodiments of the disclosure.

FIG. 20 is a rear view of another cannula configured in accordance with embodiments of the present technology.

FIGS. 21A and 21B are front isometric and rear isometric views, respectively, of a split cannula assembly configured in accordance with embodiments of the present technology.

FIGS. 22A and 22B are partially exploded isometric and partially exploded side views, respectively, of the split cannula assembly of FIG. 21A.

FIGS. 23A and 23B illustrate operation of the split cannula assembly of FIG. 21A.

FIG. 24 is an isometric view of a cannula-irrigation system configured in accordance with embodiments of the present technology.

FIG. 25 is an isometric view of a split cannula assembly configured in accordance with embodiments of the present technology.

FIG. 26 is a partially exploded isometric view of the split cannula assembly of FIG. 25.

A person skilled in the relevant art will understand that the features shown in the drawings are for purposes of illustrations, and variations, including different and/or additional features and arrangements thereof, are possible.

### DETAILED DESCRIPTION

Embodiments of the present technology are directed to medical systems, devices, and associated methods of use. At least some embodiments of a surgical system include a cannula assembly having one or more anchors or other retention features that can keep the cannula assembly at a secure position inside a patient. Additionally, or alternatively, a cannula assembly can be selectively expanded to inhibit or prevent movement of the cannula assembly inside a patient, increase the size of the working space, push apart anatomical elements, or the like. The cannula assemblies described herein can guide the delivery of implants, endoscopes, instruments, and/or other surgical devices or tools via portal sites. In spinal or other surgical procedures, precisely and securely positioning cannulas can improve patient outcomes and reduce recovery times. Certain details are set forth in the following description and in the figures to provide a thorough understanding of such embodiments of the disclosure. Other details describing well-known structures and systems often associated with, for example, surgical procedures are not set forth in the following description to avoid unnecessarily obscuring the description of various embodiments of the disclosure.

### I. OVERVIEW

At least some embodiments are directed to split cannula assemblies for use in multi-portal or other surgical procedures. For example, a split cannula assembly can include a split cannula and a retention component. The retention component can include one or more retention features such as hooks, barbs, anchors, and/or the like. The retention component can be coupled to the split cannula, and the one or more retention features can hook or otherwise grip onto patient tissue and thereby secure a position of the split cannula relative to the patient. In another example, a split cannula assembly can include a split cannula and an expander. The expander can be coupled to the split cannula and can be operated (e.g., actuated, moved) to configure the split cannula between a non-expanded state and an expanded state. Expanding the split cannula can increase the working space and help secure a position of the split cannula relative to the patient.

In some aspects, the technology relates to a split cannula assembly including a split cannula and a retention component. The split cannula can include one or more openings, and the retention component can include one or more retention features extending through corresponding ones of the one or more openings of the split cannula. The one or more retention features can be configured to anchor the split cannula at a stable position in a patient, and also can be shaped and/or movable to facilitate easy removal of the split cannula assembly from the patient. For example, the retention features can include anchors, hooks, barbs, flanges, and/or the like, and may be composed of shape memory material or be otherwise actuatable.

In some aspects, the technology relates to a split cannula assembly including a split cannula and an expander. The split cannula can include first and second legs, and the expander can be positioned at least partially between the first and second legs. The expander can be operable to move at least one of the first leg or the second leg away from the other, thereby configuring the split cannula from a non-expanded state to an expanded state. Expanding the split cannula can increase the working space and help secure a position of the split cannula relative to the patient.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

### II. MULTI-PORTAL SURGICAL SYSTEMS

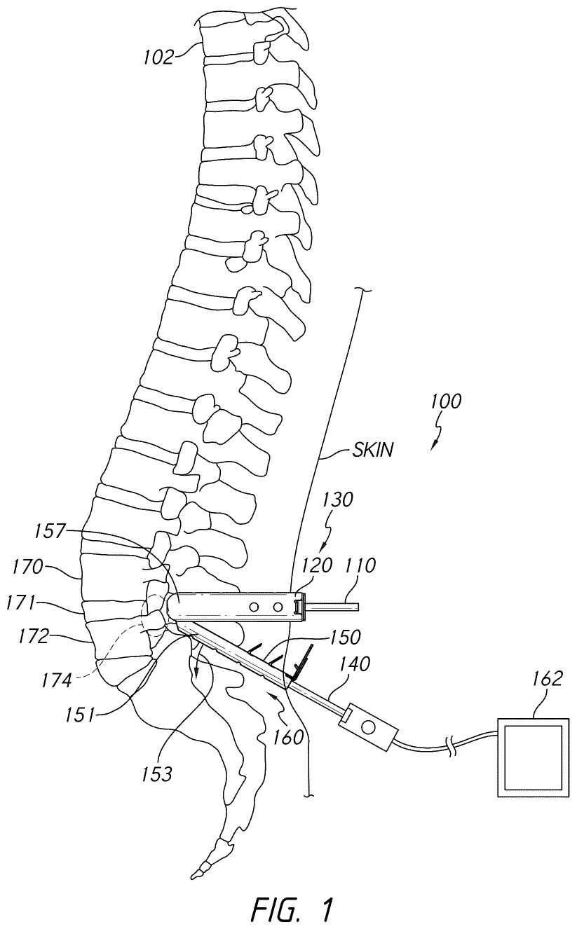

FIG. 1 is a side view of a multi-portal spinal surgical system 100 (“system 100”) positioned along a human subject's spine 102 and configured in accordance with embodiments of the disclosure. The system 100 can include an instrument assembly 130 and a visualization assembly 160. The instrument assembly 130 can include an instrument 110 and a split cannula assembly 120. The visualization assembly 160 can include a visualization instrument 140 and a split cannula assembly 150. The split cannula assemblies 120, 150 can be identical, similar to, or different from one another. The instruments 110, 140 can be moved distally and/or laterally out of the split cannula assemblies 120, 150, which can be positioned in incisions or endoscopic ports, to access a relatively large working space along the patient's spine 102. The split cannula assemblies 120, 150 can be configured to inhibit, limit, or prevent movement relative to the patient to prevent, for example, excessive penetration of the split cannula assemblies 120, 150, inadvertent withdrawal of the split cannula assemblies 120, 150 (e.g., when instruments are withdrawn from the patient), etc. The split cannula assemblies 120, 150 can have longitudinally extending openings along their entire lengths or portion thereof (not visible in FIG. 1) to allow distal portions of the respective instruments 110, 140 to be moved laterally into and out of sides of the split cannula assemblies 120, 150. The split cannula assemblies 120, 150 can be moved to repositioning configurations for repositioning (or removing) the split cannula assemblies 120, 150.

The illustrated split cannula assembly 150 has an open front side 151 (illustrated facing the inferior direction relative to the patient) through which the visualization instrument 140 can be moved, as indicated by arrow 153. The split cannula assembly 120 has an open front side (not visible in FIG. 1) facing the subject's spine such that a backside surface 157 contacts tissue. A series of instruments can be delivered through the split cannula assembly 120. In some procedures, the instrument 110 can be used to remove tissue (e.g., intervertebral disc 171, tissue contributing to stenosis, etc.), form access paths to implantation sites, prepare an implantation site by, for example, moving organs or tissue (e.g., moving nerve tissue), prepare vertebral bodies (e.g., roughening or shaping vertebral endplates), or the like. The instrument 110 can be removed and a distraction instrument (e.g., one or more dilators) can be delivered through the split cannula assembly 120 to distract adjacent vertebrae 170, 172, thereby enlarging the intervertebral space or working space 174. An interbody implant can be delivered through the split cannula assembly 120 or another cannula (e.g., a non-split tubular cannula), and into the enlarged intervertebral space. In expandable implant embodiments, an expandable interbody fusion implant can be expanded to push apart vertebral endplates.

With continued reference to FIG. 1, the visualization assembly 160 can provide intraoperative endoscopic viewing of work spaces, delivery paths, organs, tissue (e.g., nerve tissue) implantation sites, implants, interbody fusion devices (e.g., before, during, and/or after delivery), instrument(s) (including dispensers, dilators, decompression instruments, etc.), and other areas or features of interest. The position of the split cannula assemblies 120, 150 can be selected based on the procedure and optical characteristics (e.g., field of view, zoom capability, etc.) of the visualization assembly 160. The visualization assembly 160 can be moved throughout the procedure to provide intraoperative endoscopic viewing of one, multiple, or all of the surgical steps. For example, the visualization assembly 160 can be used to view tissue contributing to nerve compression caused by narrowing of the spinal canal associated with arthritis of the spine, degeneration of spinal discs, and thickening of ligaments. Arthritis of the spine often leads to the formation of bone spurs, which can narrow the spinal canal and press on the spinal cord. This tissue can be viewed using the visualization assembly 160. In spinal disc degeneration, the visualization assembly 160 can view the inner tissue of the disc protruding through a weakened fibrous outer covering of the disc and pressing on the spinal cord and/or spinal nerve roots. The protruding tissue can be viewed before and/or during removal. The visualization assembly 160 can be used to also view ligaments pressing on the spinal cord and/or nerve roots to assist in treatment.

The visualization instrument 140 can be a low-profile fiber optic endoscope positioned directly through an incision, an endoscopic port, or the like. The visualization instrument 140 can include one or more endoscopes having, without limitation, fiber optics (e.g., optical fibers), lenses, imaging devices, working lumens, light source controls, or the like for direct viewing or viewing via a display 162 (e.g., an electronic screen, monitor, etc.). In some embodiments, the visualization instrument 140 can include a lumen through which fluid flows to irrigate the surgical site. For example, saline, or another suitable liquid, can be pumped through the visualization instrument 140 to remove tissue (e.g., loose tissue, bone dust, etc.) or other material impairing visualization. The visualization instrument 140 can also include one or more lumens (e.g., irrigation return lumens, vacuum lumens, etc.) through which the irrigation liquid can be withdrawn.

The visualization instrument 140 can illuminate the body cavity and enable high-resolution video visualization. A light source (e.g., a laser, light-emitting diode, etc.) located near or at the proximal end of the fiber optics can be used to transmit light to the distal end and provide illuminating light. This enables a surgeon to safely navigate into the subject's body and to illuminate specific body anatomy to view vertebral spacing, vertebral structures, nerves, bony buildup (e.g., buildup that could be irritating and pressing against nerves contributing to nerve compression), etc. In some embodiments, visualization optics for vision and illumination are included within the distal tip of the visualization instrument 140. The configuration and functionality of the visualization instrument 140 can be selected based on the desired field of view, viewing resolution, pan/zoom functionality, or the like. Irrigation techniques, visualization devices, instruments, cannulas, and visualization and surgical techniques are discussed in U.S. application Ser. No. 17/902,685 and U.S. Pat. No. 11,678,906, which are incorporated by reference herein in their entireties.

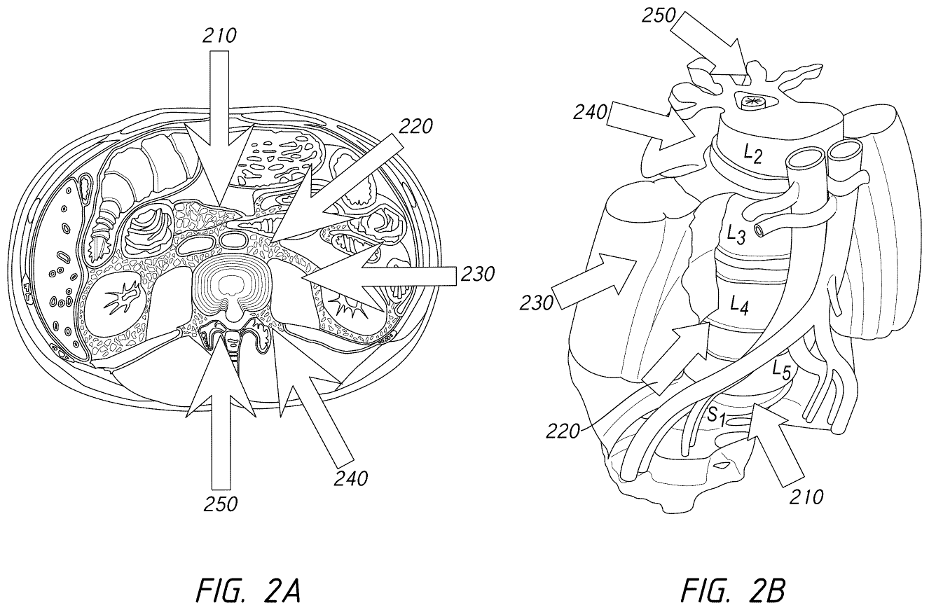

FIGS. 2A and 2B are schematic top plan and isometric views, respectively, along the lumbar spine of a human subject and illustrate example approaches for performing procedures suitable for the system 100 of FIG. 1 and other systems disclosed herein. Referring to FIGS. 2A and 2B together, surgical equipment can be delivered via different paths, including an anterior lumbar interbody fusion (ALIF) path 210, an oblique lumbar interbody fusion (OLIF) path 220, a lateral or extreme lateral lumbar interbody fusion (LLIF or XLIF) path 230, a transforaminal lumbar interbody fusion (TLIF) path 240, and a posterior lumbar interbody fusion (PLIF) path 250. These paths can also be used to perform other procedures disclosed herein. For example, one or more of the paths 210, 220, 230, 240, 250 can be selected for multi-portal endoscopic approaches to perform a wider array of lumbar spine procedures than conventional one-portal techniques. Cannulas can be positioned along the same path or different paths to allow for independent positioning and manipulation of the endoscopic camera and/or surgical instruments, thereby providing greater flexibility and enhanced visualization of spinal anatomy.

Surgical instruments can remove tissue to define working space(s) inside the patient. In one example TLIF procedure, the transforaminal path 240 may be employed to implant a single small expandable or non-expandable interbody spacer at the intervertebral space. In one example PLIF procedure, two interbody spacers can be delivered along the posterior path 250 and implanted at the intervertebral space. The two interbody spacers can cooperate to keep the vertebral bodies at the desired spacing and may be larger than the TLIF spacer. Additionally, multiple interbody spacers can provide lordotic correction by providing support at different heights. In one example LLIF procedure, a single relatively large interbody spacer can be delivered along the lateral path 230 and implanted to provide asymmetrical support. In one example ALIF procedure, an asymmetric interbody spacer can be delivered along the anterior path 210 to provide support consistent with lordosis at that portion of the spine. Lateral approaches, transforaminal approaches, and anterior approaches can be used to access the cervical spine, thoracic spine, etc. The number of instruments, configurations of instruments, implants, and surgical techniques can be selected based on the condition to be treated.

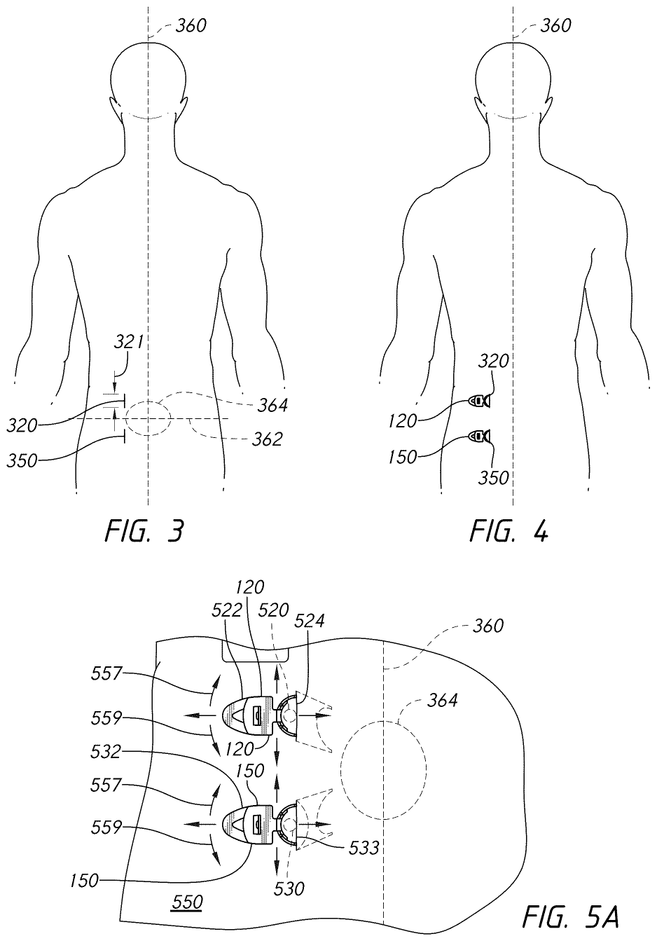

FIGS. 3-5B show steps of example multi-portal surgical procedures performed on a human subject. Multi-portal techniques can be provided to access using a wide range of different trajectories. Advantageously, tissue can rest gently across openings of the split cannula assemblies 120, 150 to inhibit, limit, or prevent movement of the split cannula assemblies 120, 150 relative to the patient when a user manually manipulates the instruments. As discussed further herein, the split cannula assemblies 120, 150 can include retention components or devices that also help provide such resistance. The instruments can also be urged against the tissue to push the instruments out of the cannulas, thereby providing flexibility to access tissue in larger working spaces. Example surgical steps are discussed below.

FIG. 3 shows two incisions 320, 350 for accessing the left side of a lumbar spine. FIG. 4 shows the split cannula assemblies 120, 150 positioned in the incisions 320, 350, respectively, for instrument insertion. The distance between the incisions 320, 350 can be equal to or less than the combined length of the cannula assemblies such that distal ends of the cannula assemblies can be moved adjacent to one another. In some embodiments, the distance between the incisions 320, 350 is equal to or less than an axial length of one, multiple, or all of the cannula assemblies (e.g., the split cannula assemblies 120, 150 in FIG. 4). The split cannula assemblies 120, 150 can be angled toward each other, as shown in FIG. 1, while maintaining a minimum distance of separation. Additional cannula assemblies or cannulas can be inserted into the subject to access other regions and/or provide alternative access paths.

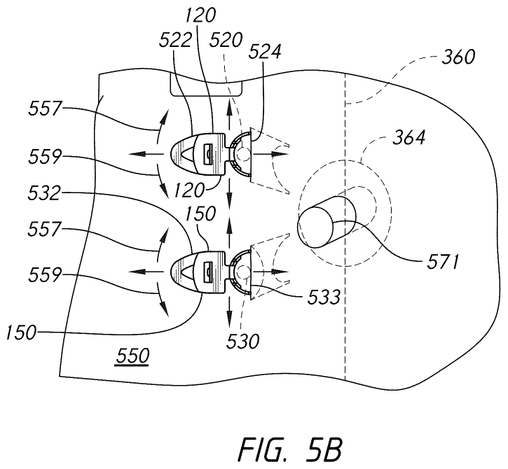

FIG. 5A shows instruments 520, 530 (illustrated as dashed circles) positioned in U-shaped passages or channels of the split cannula assemblies 120, 150. The split cannula assemblies 120, 150 and the instruments 520, 530 can be moved together or independently, as indicated by arrows 557, 559. Tissue 524, 533 extending across the open sides of the split cannula assemblies 120, 150 help keep portions of the instruments 520, 530 within the proximal ends of the split cannula assemblies 120, 150. The split cannula assemblies 120, 150 can include insertion stops, illustrated as outwardly extending flanges 522, 532, respectively, configured to contact the patient's skin 550. The split cannula assemblies 120, 150 and the instruments 520, 530, respectively, can be positioned any number of times. Additionally or alternatively, additional incisions can be made along the patient to reposition the split cannula assemblies 120, 150 or insert additional cannulas and instruments. As shown, the split cannula assemblies 120, 150 and the instruments 520, 530 can be laterally spaced apart from a target site 364 and the sagittal plane 360 therethrough.

FIG. 5B shows an optional tubular cannula 571 positioned to deliver a spinal implant, such as an intervertebral cage, interspinous spacer, screws, etc. The tubular cannula 571 can also be positioned on the opposite side of the sagittal plane 360 or at other locations. Instruments, implants, and other items can be delivered through the tubular cannula 571.

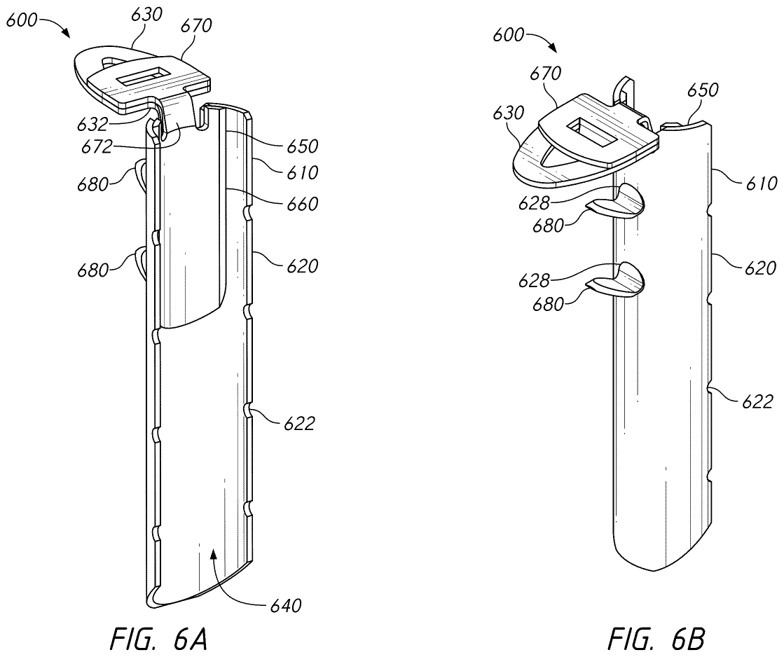

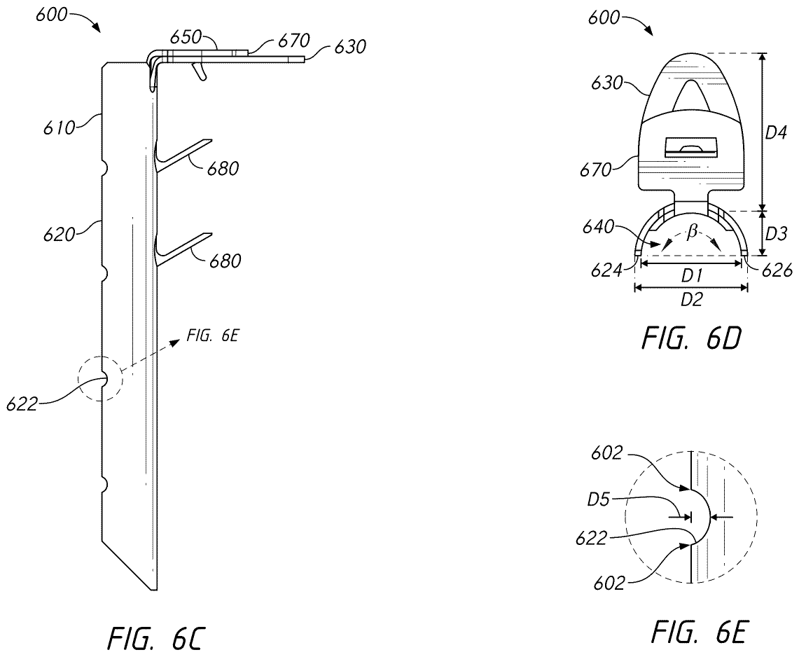

The instruments 520, 530 of FIGS. 5A and 5B can be the same as or similar to the instruments 110, 140 of FIG. 1 or other instruments disclosed herein, including instruments disclosed in U.S. application Ser. No. 17/902,685 and U.S. Pat. No. 11,678,906. Example configurations of the split cannula assemblies 120, 150 are discussed below with reference to FIGS. 6A-18.

### III. SPLIT CANNULA ASSEMBLIES

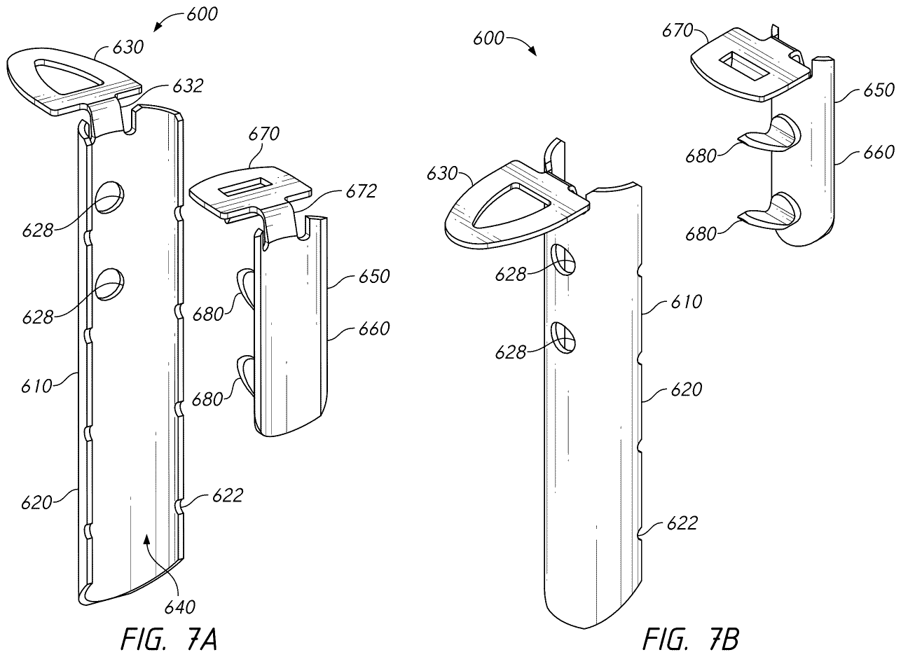

FIGS. 6A-6E are front isometric, rear isometric, side, top, and enlarged side views, respectively, of a split cannula assembly 600 (“assembly 600”) configured in accordance with embodiments of the present technology. FIGS. 7A and 7B are front partially exploded and rear partially exploded views, respectively, of the assembly 600. The assembly 600 can be an example of the split cannula assemblies 120, 150 of FIG. 1. Referring to FIGS. 6A-6E, 7A, and 7B together, the assembly 600 can include a split cannula 610 and a retention component or device 650 removably coupled to the split cannula 610.

The split cannula 610 can include an elongate split shaft 620, an insertion stop 630, and a neck portion 632 extending therebetween. The elongate split shaft 620 can have a generally U-shaped cross-section to define a U-shaped open channel 640. The elongate split shaft 620 can include a plurality of motion inhibitors 622 on the edges and along a length thereof. Referring momentarily to FIG. 6E, which is a detailed side view of one of the motion inhibitors 622, the motion inhibitor 622 of the illustrated embodiment includes a semi-circular notch with a depth D5 greater than, equal to, or less than a wall thickness of the elongate split shaft 620. In other embodiments, the motion inhibitors 622 can include a V shape, a U shape, or other shape (e.g., indentations, grooves, cutouts, ridges, protrusions) and may extend circumferentially about the elongate split shaft 620. The motion inhibitors 622 can include corners or edges 602 suitable for catching the patient's tissue (e.g., skin, shallow tissue) and thereby help secure a position of the split cannula 610 at least partially in the patient. Also, as best seen in FIGS. 7A and 7B, the elongate split shaft 620 can include one or more openings 628 (e.g., apertures) arranged along the length of the elongate split shaft 620.

The insertion stop 630 can include a flat flange oriented substantially perpendicular to the elongate split shaft 620. The neck portion 632 can have a curvature to orient the insertion stop 630 as shown. In other embodiments, the insertion stop 630 can have other shapes, sizes, and/or orientations relative to the elongate split shaft 620. In operation, when the assembly 600 is inserted into the patient by a certain depth, the insertion stop 630 can abut against the patient (e.g., against the patient's skin) and prevent further insertion of the assembly 600.

The retention device 650 can include an elongate split shaft 660, an insertion stop 670, and a neck portion 672 extending therebetween. The elongate split shaft 660 can have a generally U-shaped cross-section having a curvature or form factor corresponding to and having a smaller radius than the curvature or form factor of the elongate split shaft 620 such that the retention device 650 can be positioned in the open channel 640 and in contact with the split cannula 610. Configuring the elongate split shaft 620 and the elongate split shaft 660 to have complementary shapes can also reduce or minimize the profile of the assembly 600 while increasing or maximizing the space of the open channel 640. In the illustrated embodiment, the elongate split shaft 660 of the retention device 650 is smaller than the elongate split shaft 620 of the split cannula 610 in radius and length (e.g., by 40%, 50%, 60%, 70%, etc.). Configuring the elongate split shaft 660 to be shorter than the elongate split shaft 620 can also reduce or minimize the profile of the assembly 600 while increasing or maximizing the space of the open channel 640. In other embodiments, however, the elongate split shaft 660 can have other relative dimensions.

The insertion stop 670 can include a flat flange oriented substantially perpendicular to the elongate split shaft 660. The neck portion 672 can have a curvature to orient the insertion stop 670 as shown. In other embodiments, the insertion stop 670 can have other shapes, sizes, and/or orientations relative to the elongate split shaft 620. As shown, when the retention device 650 is coupled to the split cannula 610, the insertion stop 670 can extend over and be in contact with the insertion stop 630 of the split cannula 610. Thus, the insertion stops 630, 670 can help define the correct relative positions of the split cannula 610 and the retention device 650 when coupled together (e.g., assembled), as shown in FIGS. 6A-6D.

The retention device 650 can further include one or more retention features 680. In the illustrated embodiment, the one or more retention features 680 include two flanges each extending radially outward from the elongate split shaft 660 and angled upward (e.g., toward the insertion stop 670). Moreover, the retention features 680 are sized and positioned to extend through corresponding ones of the openings 628 when the split cannula 610 and the retention device 650 are coupled together (e.g., assembled), as shown in FIGS. 6A-6D. In other embodiments, the one or more retention features 680 can include a different number of retention features (e.g., one, three, four, five, six, or more), different dimensions (e.g., longer, shorter, wider, narrower), and/or different shapes (e.g., anchors, hooks, barbs, and/or the like). The retention features 680 can have sharp tissue-penetrating tips or edges, barbs, or other features for interacting with tissue.

Referring to FIG. 6D, the insertion stop 630 can have an axial length D4 longer than a width D1 of the open channel 640, an outer dimension D2 of the elongate split shaft 620 (e.g., a distance between outer longitudinal edges 624, 626 of the elongate split shaft 620 or an outer diameter thereof), and/or a depth D3 of the open channel 640. The width D1 can be, for example, 60 mm, 50 mm, 40 mm, 30 mm, 20 mm, or other dimensions selected based on the procedure to be performed. Because the maximum dimension of the insertion stop 630 is greater than the maximum transverse dimension of the elongate split shaft 620, the insertion stop 630 can be longer than a length of an incision in which the elongate split shaft 620 is positioned.

Also, the open channel 640 can define a sidewall with an arc length. In the illustrated embodiment, a central angle β is taken transversely along a plane generally orthogonal to the longitudinal axis of the elongate split shaft 620 and is about 180 degrees. In some embodiments, the central angle β is in a range between about 150 degrees and about 210 degrees, about 160 degrees and about 200 degrees, about 170 degrees and about 190 degrees, etc. The central angle β can be selected based on the procedure to be performed, instruments to be used, etc. Furthermore, as shown, the elongate split shaft 660 of the retention device 650 can have a central angle less than the central angle β of the open channel 640 to, e.g., keep the motion inhibitors 622 fully exposed for tissue contact. For example, the central angle of the elongate split shaft 660 can be in a range between about 75 degrees and about 105 degrees, about 80 degrees and about 100 degrees, about 85 degrees and about 95 degrees, etc.

In operation, the assembly 600 can be configured in the assembled state (FIGS. 6A-6D) prior to insertion by, e.g., moving the retention device 650 laterally toward the split cannula 610 and into the open channel 640 and/or pivoting the retention device 650 relative to the split cannula 610 to insert the retention features 680 through the corresponding openings 628. In other words, the retention device 650 can be moved from a non-anchoring position to an anchoring position in which the split cannula 610 and the retention device 650 are translationally locked. The assembly 600 can then be inserted into an incision or port in the patient, as shown in FIGS. 4-5B. Because the retention features 680 (e.g., flanges) are angled upward, the retention features 680 may not substantially impede insertion of the assembly 600 in a downward direction. Once the assembly 600 is inserted to an appropriate depth, the retention features 680 (in tandem with the motion inhibitors 622) can hook, grip, or otherwise catch onto adjacent patient tissue and thereby inhibit motion, limit motion, and/or anchor the split cannula 610 at a stable position in the patient. As one of ordinary skill in the art will appreciate, precisely and securely positioning cannulas can improve patient outcomes and reduce recovery times. Subsequently, the physician may, e.g., hold the insertion stop 670 to decouple the retention device 650 from the split cannula 610, such as by moving the retention device 650 into the open channel 640, to remove the assembly 600 from the patient.

In some embodiments, the retention features 680 are composed of Nitinol or other shape memory material. The material can be shape set so that during delivery, the retention features 680 are relatively flexible, facilitating movement of the assembly 600 inside the patient, and when at the desired depth, the retention features 680 become relatively rigid, enabling the retention features 680 to fix or otherwise secure a position of the split cannula 610 in the patient. For example, once inserted in the patient, the shape memory material can transition from a martensitic phase to an austenitic phase in response to body heat from the patient. Subsequently, a cooling fluid (e.g., cool saline) can be delivered to the assembly 600 to return the shape memory material to the martensitic phase, allowing easier removal of the assembly 600 from the patient.

In some embodiments, the retention features 680 are actuatable in other ways. For example, the retention features 680 can incorporate flanges, anchors, hooks, barbs, and/or the like that are spring-loaded to ensure secure engagement of tissue and easy insertion and removal. Alternatively or additionally, the retention features 680 may be movable or deployable through a drive mechanism, such as a motor or an actuator, allowing for precise control and positioning. Alternatively or additionally, the retention features 680 can be configured to lock into a specific position using a locking mechanism, ensuring stability in a deployed state.

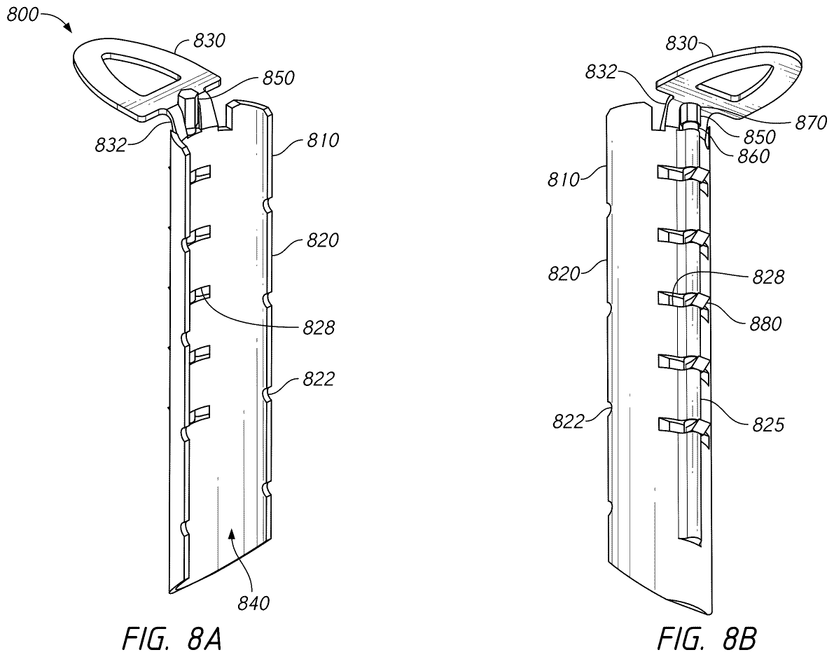

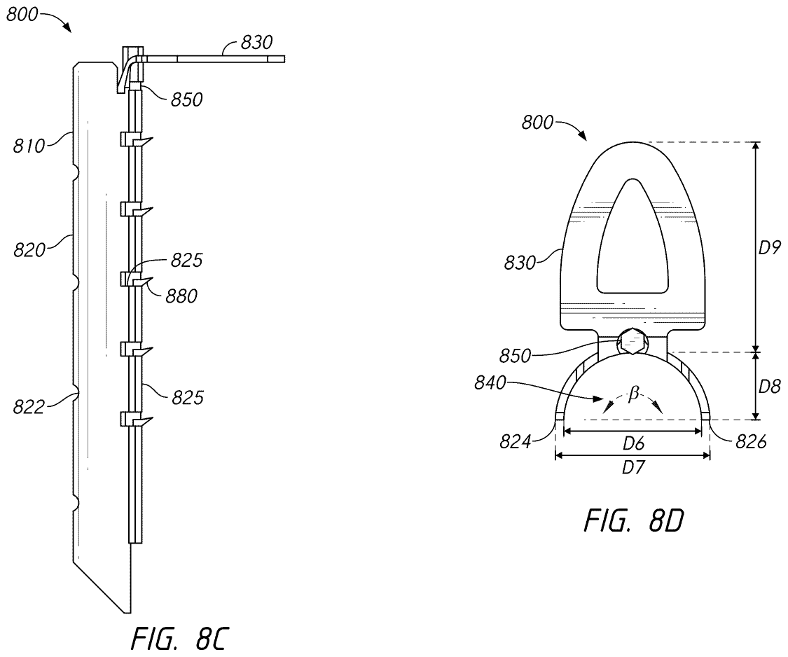

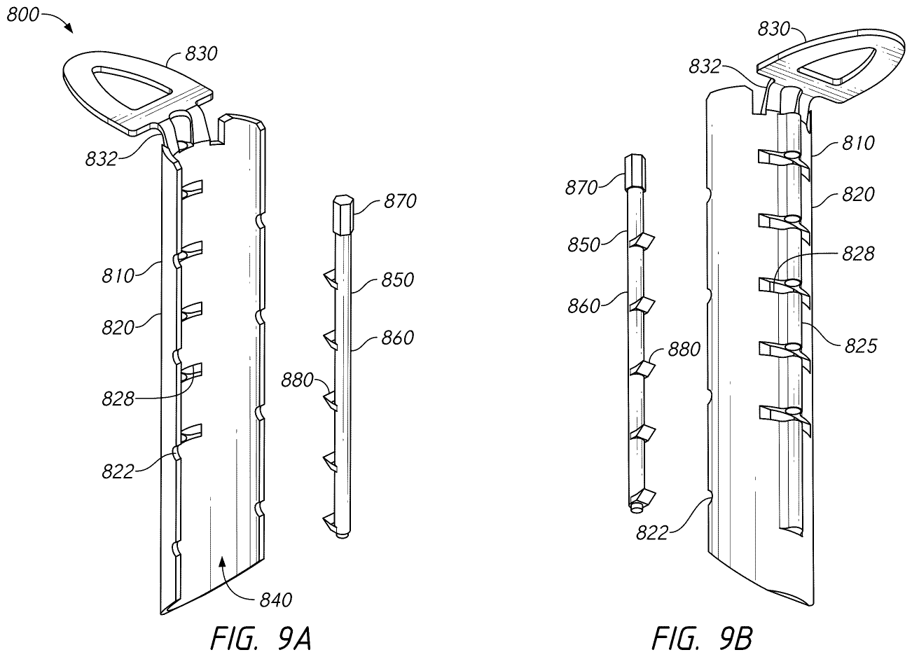

FIGS. 8A-8D are front isometric, rear isometric, side, and top views, respectively, of a split cannula assembly 800 (“assembly 800”) configured in accordance with embodiments of the present technology. FIGS. 9A and 9B are front partially exploded and rear partially exploded views, respectively, of the assembly 800. The assembly 800 can be an example of the split cannula assemblies 120, 150 of FIG. 1. Referring to FIGS. 8A-8D, 9A, and 9B together, the assembly 800 can include a split cannula 810 and a retention device 850 coupled to the split cannula 810.

The split cannula 810 can include an elongate split shaft 820, an insertion stop 830, and a neck portion 832 extending therebetween. The elongate split shaft 820 can have a generally U-shaped cross-section to define a U-shaped open channel 840. The elongate split shaft 820 can include a plurality of motion inhibitors 822 on the edges and along a length thereof. The motion inhibitors 822 can be identical or substantially similar to the motion inhibitor 622 illustrated in FIG. 6E. The elongate split shaft 820 can also include one or more tubular portions or holders 825 on a rear side thereof and one or more openings 828 (e.g., slots) arranged along the length of the elongate split shaft 820. The tubular holders 825 can define channels extending therethrough for receiving the retention device 850. Also, each of the openings 828 can align with a corresponding one of the gaps between adjacent ones of the tubular holders 825.

The insertion stop 830 can include a flat flange oriented substantially perpendicular to the elongate split shaft 820. The neck portion 832 can have a curvature to orient the insertion stop 830 as shown. In other embodiments, the insertion stop 830 can have other shapes, sizes, and/or orientations relative to the elongate split shaft 820. In operation, when the assembly 800 is inserted into the patient by a certain depth, the insertion stop 830 can abut against the patient (e.g., against the patient's skin) and prevent further insertion of the assembly 800.

The retention device 850 can include an elongate rod 860, a knob 870 coupled to the elongate rod 860, and one or more retention features 880 coupled to the elongate rod 860. The elongate rod 860 can be positioned to extend through the channels of the tubular holders 825 and can be rotatable about a longitudinal axis of the elongate rod 860 therein. The elongate rod 860 can be shorter than, equal in length to, or longer than the elongate split shaft 820 of the split cannula 810. The knob 870 can be coupled to an upper end portion of the elongate rod 860 and can be used to rotate the retention device 850. In the illustrated embodiment, the knob 870 has a hexagonal cross-section that facilitates gripping by a physician. The knob 870 can have other shapes.

As best seen in FIGS. 9A and 9B, the retention features 880 include a plurality of flanges (e.g., five flanges), each with a lateral upper surface and an angled lower surface, and extending radially outward from the elongate rod 860. The retention features 880 can be coupled to and arranged along the length of one side of the elongate rod 860. Moreover, the retention features 880 are sized and spaced apart from one another to fit through corresponding ones of the openings 828 of the elongate split shaft 820, as shown in FIGS. 8A-8D. In other embodiments, the one or more retention features 880 can include a different number of retention features (e.g., one, two, three, four, six, or more), different dimensions (e.g., longer, shorter, wider, narrower), and/or different shapes (e.g., anchors, hooks, barbs, and/or the like).

Referring again to FIG. 8D, the insertion stop 830 can have an axial length D9 longer than a width D6 of the open channel 840, an outer dimension D7 of the elongate split shaft 820 (e.g., a distance between outer longitudinal edges 824, 826 of the elongate split shaft 820 or an outer diameter thereof), and/or a depth D8 of the open channel 840. The width D6 can be, for example, 60 mm, 50 mm, 40 mm, 30 mm, 20 mm, or other dimensions selected based on the procedure to be performed. Because the maximum dimension of the insertion stop 830 is greater than the maximum transverse dimension of the elongate split shaft 820, the insertion stop 830 can be longer than a length of an incision in which the elongate split shaft 820 is positioned.

Also, the open channel 840 can define a sidewall with an arc length. In the illustrated embodiment, a central angle β is taken transversely along a plane generally orthogonal to the longitudinal axis of the elongate split shaft 820 and is about 180 degrees. In some embodiments, the central angle β is in a range between about 150 degrees and about 210 degrees, about 160 degrees and about 200 degrees, about 170 degrees and about 190 degrees, etc. The central angle β can be selected based on the procedure to be performed, instruments to be used, etc. Furthermore, as shown, the retention device 850 can be positioned and oriented to not extend into the open channel 840.

In operation, the assembly 800 can be configured in the assembled state (FIGS. 8A-8D) prior to insertion. In particular, the retention device 850 can be oriented such that the retention features 880 face away from the open channel 840, protrude radially outward, and extend laterally beyond the tubular holders 825 (as best seen in FIG. 8C). In other words, the retention device 850 can be moved between positions, such as from a non-anchoring position to an anchoring position. Then, the assembly 800 can be inserted into an incision or port in the patient, as shown in FIGS. 4-5B. Because the retention features 880 (e.g., flanges) have angled lower surfaces, the retention features 880 may not substantially impede insertion of the assembly 800 in a downward direction. Once the assembly 800 is inserted to an appropriate depth, because the retention features 880 have lateral upper surfaces, the retention features 880 (in tandem with the motion inhibitors 822) can hook, grip, or otherwise catch onto adjacent patient tissue and thereby anchor the split cannula 810 at a stable position in the patient. As one of ordinary skill in the art will appreciate, precisely and securely positioning cannulas can improve patient outcomes and reduce recovery times. Subsequently, the physician may, e.g., use the knob 870 to rotate the retention device 850 such that the retention features 880 face sideways in the openings 828 or into the open channel 840, and do not protrude beyond the tubular holders 825, to remove the assembly 800 from the patient.

In some embodiments, the retention features 880 are composed of Nitinol or other shape memory material. The material can be shape set so that during delivery, the retention features 880 are relatively flexible, facilitating movement of the assembly 800 inside the patient, and when at the desired depth, the retention features 880 become relatively rigid, enabling the retention features 880 to fix or otherwise secure a position of the split cannula 810 in the patient. For example, once inserted in the patient, the shape memory material can transition from a martensitic phase to an austenitic phase in response to body heat from the patient. Subsequently, a cooling fluid (e.g., cool saline) can be delivered to the assembly 800 to return the shape memory material to the martensitic phase, allowing easier removal of the assembly 800 from the patient.

In some embodiments, the retention features 880 are actuatable in other ways. For example, the retention features 880 can incorporate one or more flanges, anchors, hooks, barbs, and/or the like that are spring-loaded to ensure secure engagement of tissue and easy insertion and removal. Alternatively or additionally, the retention features 880 may be movable or deployable through a drive mechanism, such as a motor or an actuator, allowing for precise control and positioning. Alternatively or additionally, the retention features 880 can be configured to lock into a specific position using a locking mechanism, ensuring stability in a deployed state.

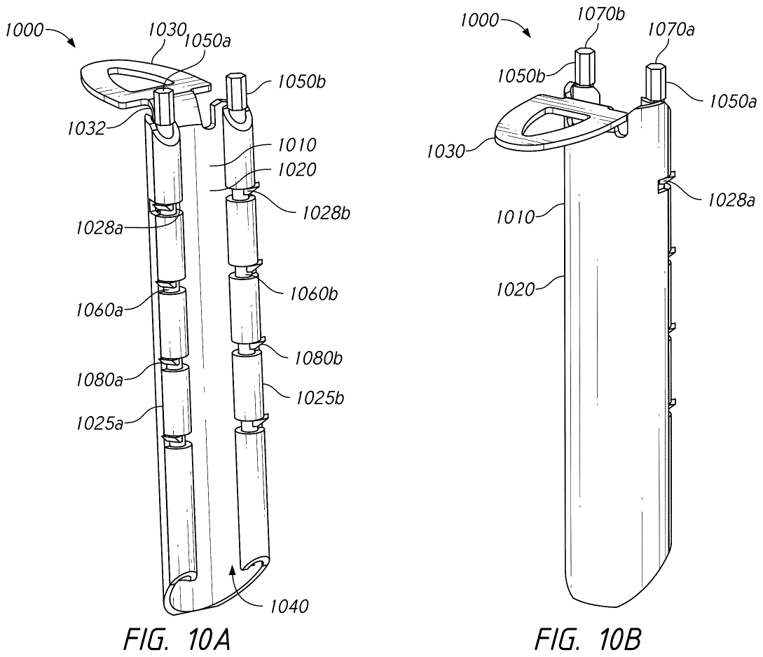

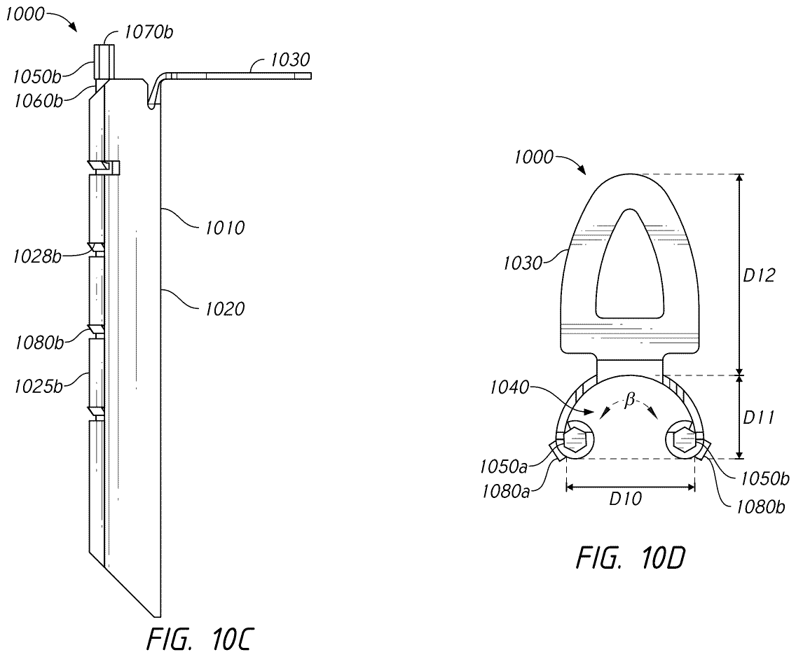

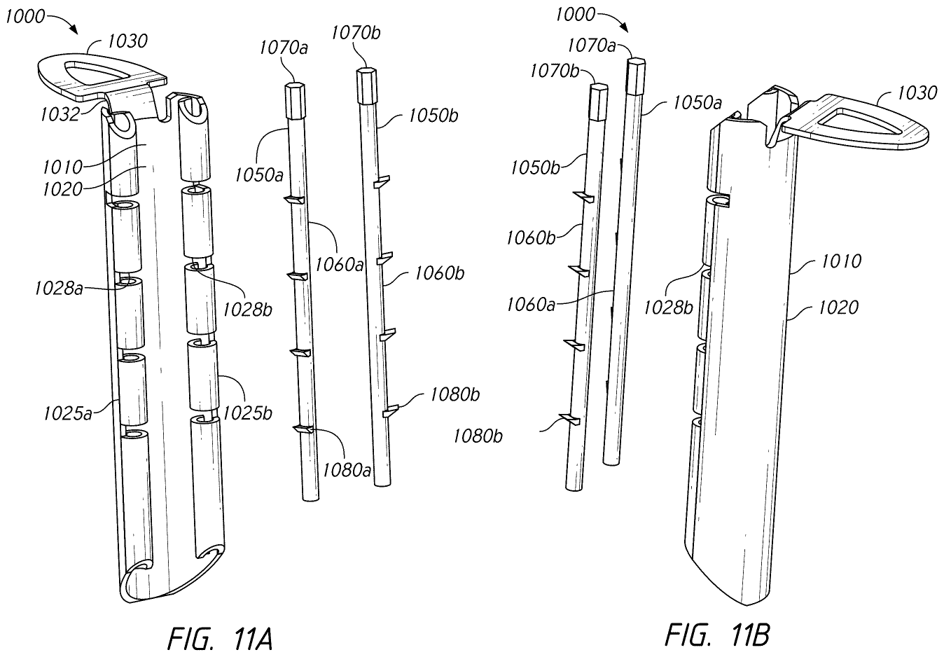

FIGS. 10A-10D are front isometric, rear isometric, side, and top views, respectively, of a split cannula assembly 1000 (“assembly 1000”) configured in accordance with embodiments of the present technology. FIGS. 11A and 11B are front partially exploded and rear partially exploded views, respectively, of the assembly 1000. The assembly 1000 can be an example of the split cannula assemblies 120, 150 of FIG. 1. Referring to FIGS. 10A-10D, 11A, and 11B together, the assembly 1000 can include a split cannula 1010, a first retention device 1050a coupled to the split cannula 1010, and a second retention device 1050b coupled to the split cannula 1010.

The split cannula 1010 can include an elongate split shaft 1020, an insertion stop 1030, and a neck portion 1032 extending therebetween. The elongate split shaft 1020 can have a generally U-shaped cross-section to define a U-shaped open channel 1040. The elongate split shaft 1020 can include one or more right or first tubular portions or holders 1025a on a front and first side (e.g., right side) thereof and one or more left or second tubular holders 1025b on a front and second side (e.g., left side) thereof. The first tubular holders 1025a and the second tubular holders 1025b can define channels extending therethrough for receiving the first retention device 1050a and the second retention device 1050b, respectively.

The insertion stop 1030 can include a flat flange oriented substantially perpendicular to the elongate split shaft 1020. The neck portion 1032 can have a curvature to orient the insertion stop 1030 as shown. In other embodiments, the insertion stop 1030 can have other shapes, sizes, and/or orientations relative to the elongate split shaft 1020. In operation, when the assembly 1000 is inserted into the patient by a certain depth, the insertion stop 1030 can abut against the patient (e.g., against the patient's skin) and prevent further insertion of the assembly 1000.

Each of the first and second retention devices 1050a, 1050b can include an elongate rod 1060a, 1060b, a knob 1070a, 1070b coupled to the elongate rod 1060a, 1060b, and one or more retention features 1080a, 1080b coupled to the elongate rod 1060a, 1060b. The elongate rod 1060a, 1060b can be positioned to extend through the channels of the tubular holders 1025a, 1025b and can be rotatable about a longitudinal axis of the elongate rod 1060a, 1060b therein. The elongate rod 1060a, 1060b can be shorter than, equal in length to, or longer than the elongate split shaft 1020 of the split cannula 1010. The knob 1070a, 1070b can be coupled to an upper end portion of the elongate rod 1060a, 1060b and can be used to rotate the retention device 1050a, 1050b. In the illustrated embodiment, the knob 1070a, 1070b has a hexagonal cross-section that facilitates gripping by a physician. The knob 1070a, 1070b can have other shapes.

As best seen in FIGS. 11A and 11B, each of the first and second retention features 1080a, 1080b include four flanges, each with a lateral upper surface and an angled lower surface, and extending radially outward from the elongate rod 1060a, 1060b. The retention features 1080a, 1080b can be coupled to and arranged along the length of one side of the elongate rod 1060a, 1060b. Moreover, the retention features 1080a, 1080b are sized and spaced apart from one another to fit through corresponding ones of gaps 1028a, 1028b between adjacent ones of the tubular holders 1025a, 1025b of the elongate split shaft 1020, as shown in FIGS. 10A-10D. In other embodiments, the one or more retention features 1080a, 1080b can include a different number of retention features (e.g., one, two, three, five, six, or more), different dimensions (e.g., longer, shorter, wider, narrower), and/or different shapes (e.g., anchors, hooks, barbs, and/or the like).

Referring to FIG. 10D, the insertion stop 1030 can have an axial length D12 longer than a width D10 of the open channel 1040 and/or a depth D11 of the open channel 1040. The width D10 can be, for example, 60 mm, 50 mm, 40 mm, 30 mm, 20 mm, or other dimensions selected based on the procedure to be performed. Because the maximum dimension of the insertion stop 1030 is greater than the maximum transverse dimension of the elongate split shaft 1020, the insertion stop 1030 can be longer than a length of an incision in which the elongate split shaft 1020 is positioned.

Also, the open channel 1040 can define a sidewall with an arc length. In the illustrated embodiment, a central angle β is taken transversely along a plane generally orthogonal to the longitudinal axis of the elongate split shaft 1020 and is about 180 degrees. In some embodiments, the central angle β is in a range between about 150 degrees and about 210 degrees, about 160 degrees and about 200 degrees, about 170 degrees and about 190 degrees, etc. The central angle β can be selected based on the procedure to be performed, instruments to be used, etc. Furthermore, as shown, the first and second retention devices 1050a, 1050b can be positioned and oriented to not extend into the open channel 1040.

In operation, the assembly 1000 can be configured in the assembled state (FIGS. 10A-10D) prior to insertion. In particular, the first and second retention devices 1050a, 1050b can be oriented such that the retention features 1080a, 1080b face away from the open channel 1040, protrude radially outward, and extend laterally beyond the tubular holders 1025a, 1025b (as best seen in FIG. 10A). In other words, the retention devices 1050a, 1050b can be moved from a non-anchoring position to an anchoring position. Then, the assembly 1000 can be inserted into an incision or port in the patient, as shown in FIGS. 4-5B. Because the retention features 1080a, 1080b (e.g., flanges) have angled lower surfaces, the retention features 1080a, 1080b may not substantially impede insertion of the assembly 1000 in a downward direction. Once the assembly 1000 is inserted to an appropriate depth, because the retention features 1080a, 1080b have lateral upper surfaces, the retention features 1080a, 1080b can hook, grip, or otherwise catch onto adjacent patient tissue and thereby anchor the split cannula 1010 at a stable position in the patient. As one of ordinary skill in the art will appreciate, precisely and securely positioning cannulas can improve patient outcomes and reduce recovery times. Subsequently, the physician may, e.g., use the knobs 1070a, 1070b to rotate corresponding ones of the retention devices 1050a, 1050b such that the retention features 1080a, 1080b face into the open channel 1040, and do not protrude beyond the tubular holders 1025a, 1025b, to remove the assembly 1000 from the patient.

In some embodiments, the retention features 1080a, 1080b are composed of Nitinol or other shape memory material. The material can be shape set so that during delivery, the retention features 1080a, 1080b are relatively flexible, facilitating movement of the assembly 1000 inside the patient, and when at the desired depth, the retention features 1080a, 1080b become relatively rigid, enabling the retention features 1080a, 1080b to fix or otherwise secure a position of the split cannula 1010 in the patient. For example, once inserted in the patient, the shape memory material can transition from a martensitic phase to an austenitic phase in response to body heat from the patient. Subsequently, a cooling fluid (e.g., cool saline) can be delivered to the assembly 1000 to return the shape memory material to the martensitic phase, allowing easier removal of the assembly 1000 from the patient.

In some embodiments, the retention features 1080a, 1080b are actuatable in other ways. For example, the retention features 1080a, 1080b can incorporate one or more flanges, anchors, hooks, barbs, and/or the like that are spring-loaded to ensure secure engagement of tissue and easy insertion and removal. Alternatively or additionally, the retention features 1080a, 1080b may be movable or deployable through a drive mechanism, such as a motor or an actuator, allowing for precise control and positioning. Alternatively or additionally, the retention features 1080a, 1080b can be configured to lock into a specific position using a locking mechanism, ensuring stability in a deployed state.

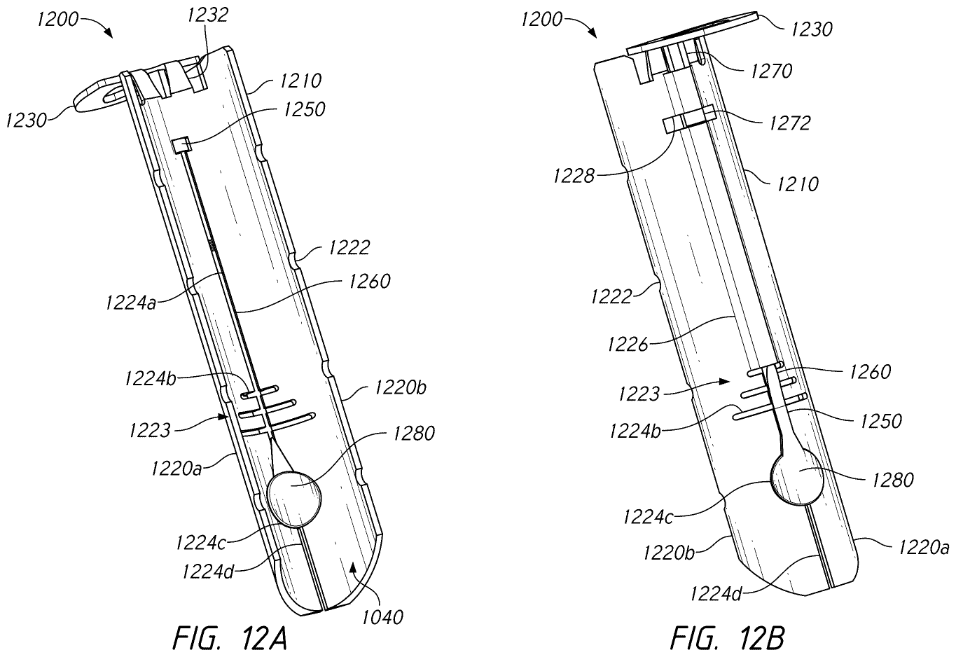

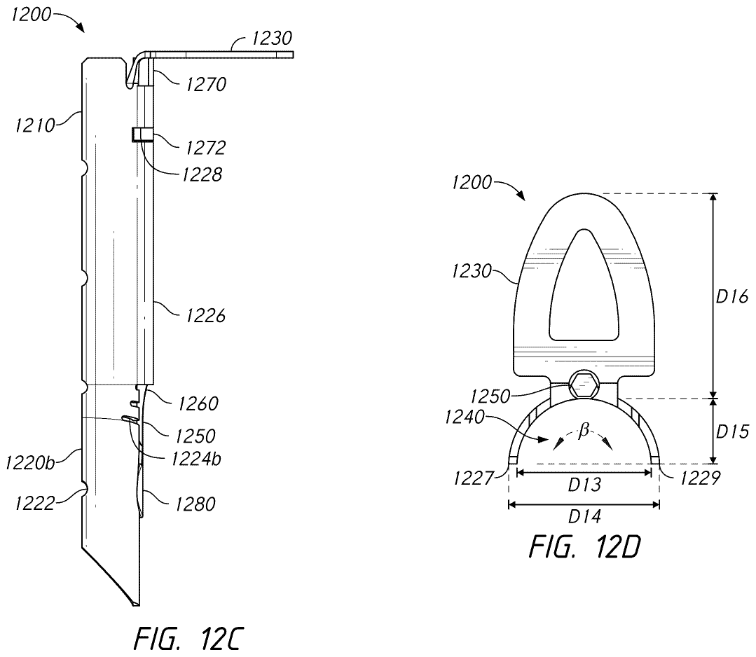

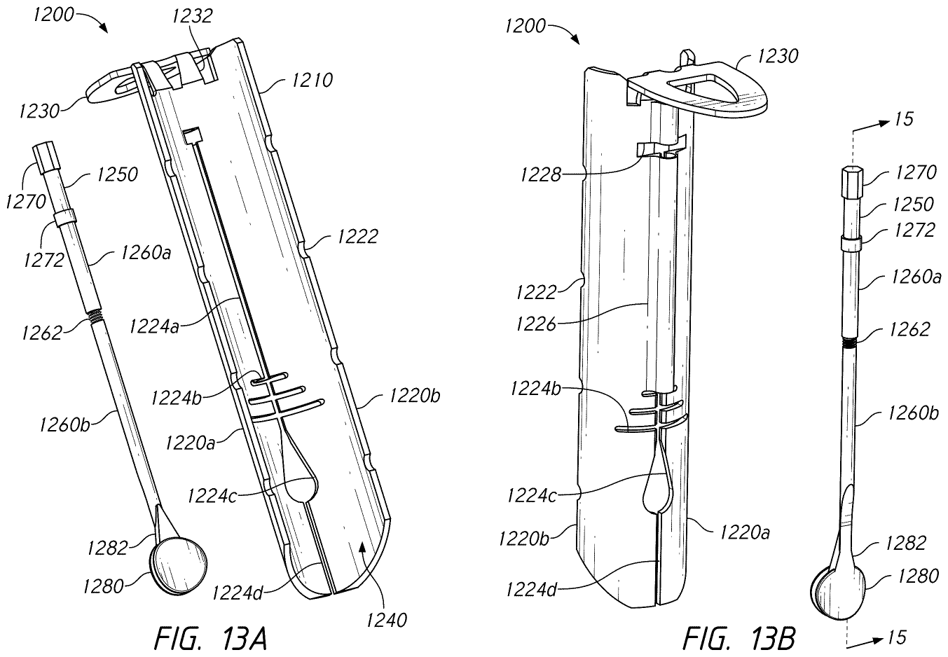

FIGS. 12A-12D are front isometric, rear isometric, side, and top views, respectively, of a split cannula assembly 1200 (“assembly 1200”) configured in accordance with embodiments of the present technology. FIGS. 13A and 13B are front partially exploded and rear partially exploded views, respectively, of the assembly 1200. The assembly 1200 can be an example of the split cannula assemblies 120, 150 of FIG. 1. Referring to FIGS. 12A-12D, 13A, and 13B together, the assembly 1200 can include a split cannula 1210 and an expander 1250 coupled to the split cannula 1210.

The split cannula 1210 can include a first leg 1220a and a second leg 1220b that form an elongate split shaft, an insertion stop 1230, and a neck portion 1232 extending therebetween. The first and second legs 1220a, 1220b can be connected at an upper portion of the split cannula 1210, but separated along the remainder of the length of the split cannula 1210 by a biasing section 1223 (FIGS. 12A and 12B). In some embodiments, the biasing section 1223 can include a plurality of slots, elongate members, cutouts, or other features that allow movement of the first and second legs 1220a, 1220b. In the illustrated embodiment, the biasing section 1223 includes an elongate slot 1224a, one or more lateral slots 1224b, a teardrop-shaped opening or gap 1224c, and a distal slot 1224d.

The elongate slot 1224a can extend from the upper portion of the split cannula 1210 to the teardrop-shaped gap 1224c, along a length of the split cannula 1210. The one or more lateral slots 1224b extend substantially perpendicular to the elongate slot 1224a and along the curvature of the split cannula 1210. The one or more lateral slots 1224b can have different lengths, as shown, or the same length as one another. The teardrop-shaped gap 1224c can extend between the one or more lateral slots 1224b and the distal slot 1224d. The teardrop-shaped gap 1224c can be relatively large in size such that, as discussed in further detail below, portions of the expander 1250 can fit therein. In other embodiments, the teardrop-shaped gap 1224c can have other shapes or form factors. The distal slot 1224d can extend from the teardrop-shaped gap 1224c until the distal end of the split cannula 1210. Therefore, by virtue of the elongate slot 1224a, the one or more lateral slots 1224b, the teardrop-shaped gap 1224c, and the distal slot 1224d, the first and second legs 1220a, 1220b include separate, cantilevered legs that are supported and connected at the upper portion of the split cannula 1210.

The split cannula 1210 can have sufficient material stiffness such that the first and second legs 1220a, 1220b form the elongate split shaft having a generally U-shaped cross-section to define a U-shaped open channel 1240. Each of the first and second legs 1220a, 1220b can include a plurality of motion inhibitors 1222 on the edges and along a length thereof. The motion inhibitors 1222 can be identical or substantially similar to the motion inhibitor 622 illustrated in FIG. 7. The split cannula 1210 can also include one or more tubular portions 1226 on a rear side thereof and one or more openings 1228 (e.g., slots) arranged along the length of the split cannula 1210. In the illustrated embodiment, the split cannula 1210 includes two tubular portions 1226 and a single gap or opening 1228 therebetween. In particular, the tubular portion 1226 extending below the opening 1228 extends behind and along the elongate slot 1224a such that the first and second legs 1220a, 1220b may not be separated along the elongate slot 1224a. The tubular portions 1226 can define channels extending therethrough for receiving the expander 1250.

The insertion stop 1230 can include a flat flange oriented substantially perpendicular to the first and second legs 1220a, 1220b. The neck portion 1232 can have a curvature to orient the insertion stop 1230 as shown. In other embodiments, the insertion stop 1230 can have other shapes, sizes, and/or orientations relative to the elongate split shaft. In operation, when the assembly 1200 is inserted into the patient by a certain depth, the insertion stop 1230 can abut against the patient (e.g., against the patient's skin) and prevent further insertion of the assembly 1200.

The expander 1250 can include a first elongate portion 1260a, a second elongate portion 1260b, a wedge-shaped portion 1282, a head or disc-shaped portion 1280, a notch 1272, and a knob 1270. The first and second elongate portions 1260a, 1260b can be positioned to extend through the channels of the tubular portions 1226. Also, the first elongate portion 1260a can be rotatable about a longitudinal axis of the first elongate portion 1260a. The expander 1250 can be shorter than, equal in length to, or longer than the split cannula 1210. The second elongate portion 1260b can include threads 1262 and can be in threaded engagement with the first elongate portion 1260a. The wedge-shaped portion 1282 can be coupled to a lower end portion of the second elongate portion 1260b, and the disc-shaped portion 1280 can be coupled to a lower end portion of the wedge-shaped portion 1282. As best seen by comparing FIGS. 12A and 13A, the wedge-shaped portion 1282 and the disc-shaped portion 1280 can be shaped and sized to generally fit in the teardrop-shaped gap 1224c.

The knob 1270 can be coupled to an upper end portion of the first elongate portion 1260a, positioned above the upper tubular portion 1226, and can be used to rotate the first elongate portion 1260a. In the illustrated embodiment, the knob 1270 has a hexagonal cross-section that facilitates gripping by a physician. The knob 1270 can have other shapes. The notch 1272 can include an annular component coupled to the first elongate portion 1260a along its length. The notch 1272 can be positioned in the opening 1228 and between the two tubular portions 1226.

Referring to FIG. 12D, the insertion stop 1230 can have an axial length D16 longer than a width D13 of the open channel 1240, an outer dimension D14 of the elongate split shaft (e.g., a distance between outer longitudinal edges 1227, 1229 of the elongate split shaft or an outer diameter thereof), and/or a depth D15 of the open channel 1240. The width D13 can be, for example, 60 mm, 50 mm, 40 mm, 30 mm, 20 mm, or other dimensions selected based on the procedure to be performed. Because the maximum dimension of the insertion stop 1230 is greater than the maximum transverse dimension of the elongate split shaft, the insertion stop 1230 can be longer than a length of an incision in which the elongate split shaft is positioned.

Also, the open channel 1240 can define a sidewall with an arc length. In the illustrated embodiment, a central angle β is taken transversely along a plane generally orthogonal to the longitudinal axis of the elongate split shaft and is about 180 degrees. In some embodiments, the central angle β is in a range between about 150 degrees and about 210 degrees, about 160 degrees and about 200 degrees, about 170 degrees and about 190 degrees, etc. The central angle β can be selected based on the procedure to be performed, instruments to be used, etc.

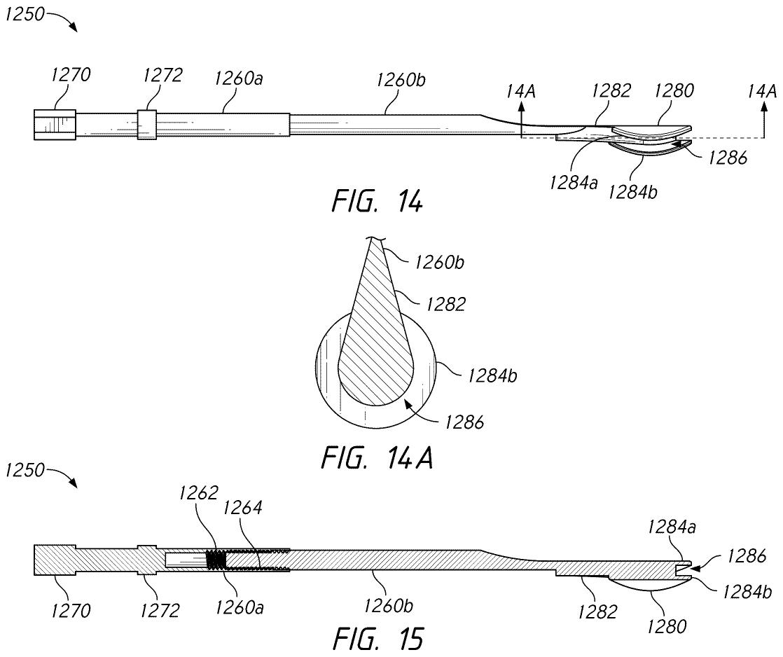

FIG. 14 is a side view of the expander 1250. FIG. 14A is a cross-sectional view of the expander 1250 taken along line 14A-14A of FIG. 14. FIG. 15 is a side cross-sectional view of the expander 1250 taken along line 15-15 of FIG. 13B. Referring to FIGS. 14, 14A, and 15 together, in the illustrated embodiment, the disc-shaped portion 1280 includes a first lip portion 1284a and a second lip portion 1284b spaced apart from the first lip portion 1284a. The first and second lip portions 1284a, 1284b can define an annular gap 1286 therebetween (best seen in FIG. 14A). Each of the first and second lip portions 1284a, 1284b can have a generally concave circular or disc shape, and the concave curvatures of the first and second lip portions 1284a, 1284b can correspond to the curvatures of the first and second legs 1220a, 1220b that form the elongate split shaft. The corresponding curvatures or other form factors can help minimize the profile of the assembly 1200 and maximize the space of the open channel 1240. Moreover, as seen in FIG. 15, the first elongate portion 1260a can have threads 1264 that can engage the threads 1262 of the second elongate portion 1260b.

In operation, the assembly 1200 can be inserted into a port or incision in a patient while the split cannula 1210 and the expander 1250 are assembled and in a non-expanded state, as shown in FIGS. 12A-12D. When in the non-expanded state, the wedge-shaped portion 1282 and the disc-shaped portion 1280 can be positioned in the teardrop-shaped gap 1224c, and portions of the first and second legs 1220a, 1220b can be partially in the gap 1286 between the first and second lip portions 1284a, 1284b of the disc-shaped portion 1280. Thus, the first lip portion 1284a can be positioned on front sides of the first and second legs 1220a, 1220b (e.g., in the open channel 1240), and the second lip portion 1284b can be positioned on rear sides thereof. Moreover, the first and second legs 1220a, 1220b extend linearly, parallel, and close to one another, defining the non-expanded state of the assembly 1200.

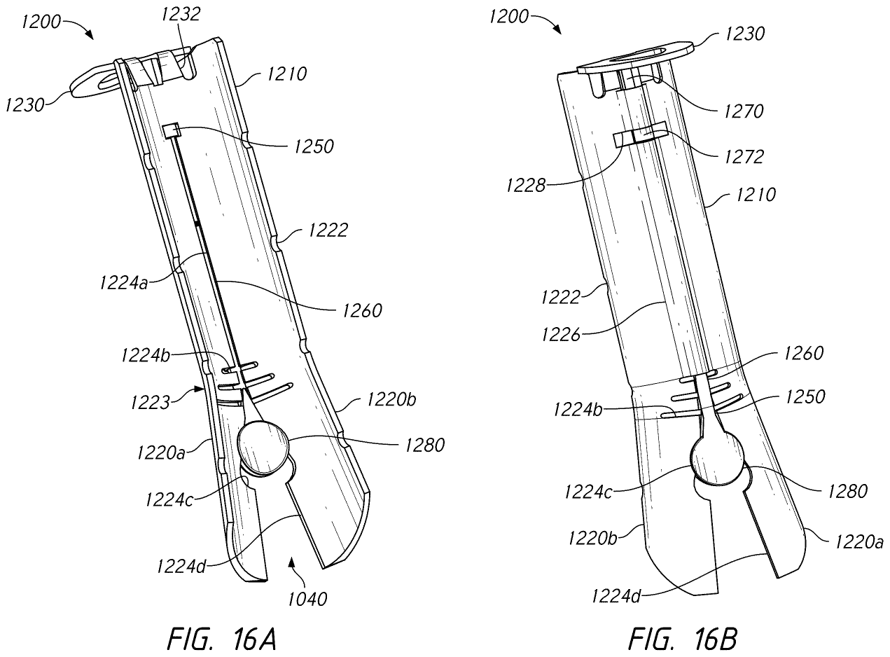

Once the assembly 1200 is inserted to a desired depth in patient tissue, the assembly 1200 can be expanded by operating the expander 1250. FIGS. 16A and 16B are front and rear views, respectively, of the assembly 1200 in an expanded state. As shown, the first and second legs 1220a, 1220b are pushed outward and away from one another, enlarging the space of the open channel 1240. Also, the wedge-shaped portion 1282 and the disc-shaped portion 1280 have moved upward and offset from the teardrop-shaped gap 1224c. Portions of the first and second legs 1220a, 1220b can remain partially in the gap 1286 between the first and second lip portions 1284a, 1284b of the disc-shaped portion 1280.

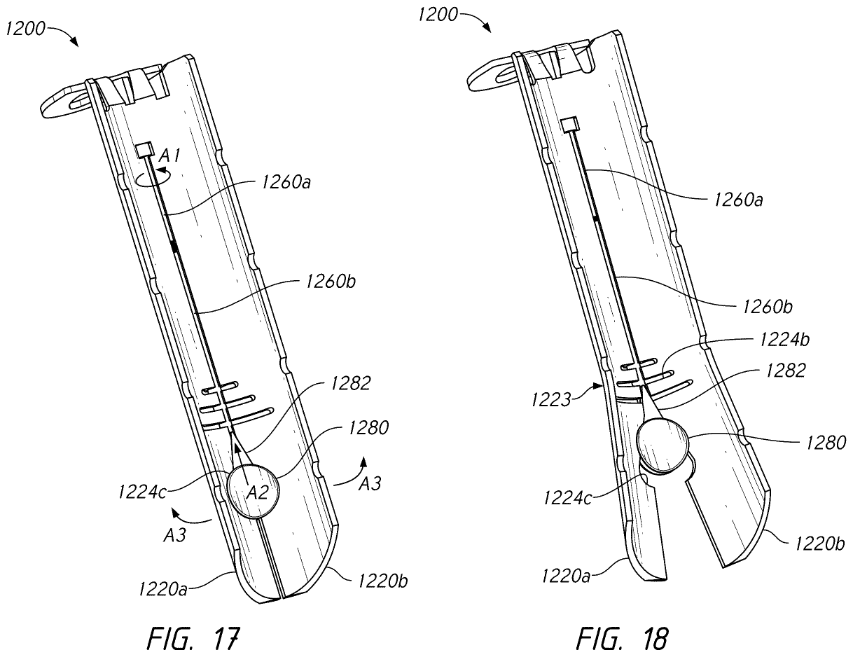

FIGS. 17 and 18 illustrate expansion of the assembly 1200. Specifically, FIG. 17 illustrates the assembly 1200 in the non-expanded state with arrows indicating the motions associated with configuring the assembly 1200 to the expanded state, and FIG. 18 illustrates the assembly 1200 in the expanded state.

Referring to FIG. 17, once the assembly 1200 is inserted to an appropriate depth, the physician can rotate the knob 1270 (as indicated by arrow A1) to configure the assembly 1200 from the non-expanded state to the expanded state. More specifically, as the knob 1270 (and the first elongate portion 1260a fixedly attached thereto) is rotated, the notch 1272 positioned in the opening 1228 between the two tubular portions 1226 (FIG. 12B) prevents axial movement of the first elongate portion 1260a. Also, the portions of the first and second legs 1220a, 1220b positioned in the gap 1286 of the expander 1250 prevent rotation of the second elongate portion 1260b. Thus, rotating the knob 1270 rotates the first elongate portion 1260a (arrow A1) at a fixed axial position relative to the split cannula 1210, and allows the threads 1262 to rotate with respect to the threads 1264, thereby moving the second elongate portion 1260b in a proximal direction (as indicated by arrow A2) and without rotating relative to the split cannula 1210.

As the second elongate portion 1260b moves upward (arrow A2), the wedge-shaped portion 1282 pushes against the inner sides of the first and second legs 1220a, 1220b, gradually spreading them apart (as indicated by arrows A3). The lateral slots 1224b can facilitate the spreading of the first and second legs 1220a, 1220b, as the material forming the split cannula 1210 can be relatively stiff. Thus, when the knob 1270 is sufficiently rotated, the assembly 1200 is configured as shown in FIG. 18. The portions of the first and second legs 1220a, 1220b along the elongate slot 1224a can remain substantially unexpanded due to the tubular portions 1226 keeping the first and second legs 1220a, 1220b together, while the portions of the first and second legs 1220a, 1220b along the teardrop-shaped gap 1224c and the distal slot 1224d are spread apart to define the expanded state of the assembly 1200. Expanding the profile of the assembly 1200 as illustrated and described herein is expected to increase the working space and help keep the split cannula 1210 in a fixed position and orientation with respect to the patient (in tandem with the motion inhibitors 1222) during operation. Moreover, because the wedge-shaped portion 1282 maintains contact with the inner sides of the first and second legs 1220a, 1220b, and portions of the first and second legs 1220a, 1220b remain in the gap 1286 of the expander 1250, the assembly 1200 avoids forming any significant gaps between the split cannula 1210 and the expander 1250 in which patient tissue may be cinched and damaged.

After use, the physician can remove the assembly 1200 by first returning the assembly to the non-expanded state. For example, the physician can rotate the knob 1270 (in the opposite direction of arrow A1) such that the second elongate portion 1260b moves back downward (in the opposite direction of arrow A2). The material forming the split cannula 1210 can be relatively stiff such that the first and second legs 1220a, 1220b are biased toward one another, and returning the wedge-shaped portion 1282 and the disc-shaped portion 1280 to their respective positions in the teardrop-shaped gap 1224c can allow the first and second legs 1220a, 1220b to move back toward one another (in the opposite direction of arrows A3). Returning the assembly 1200 to the non-expanded state and thereby reducing the profile of the assembly 1200 can facilitate removal thereof from the patient.

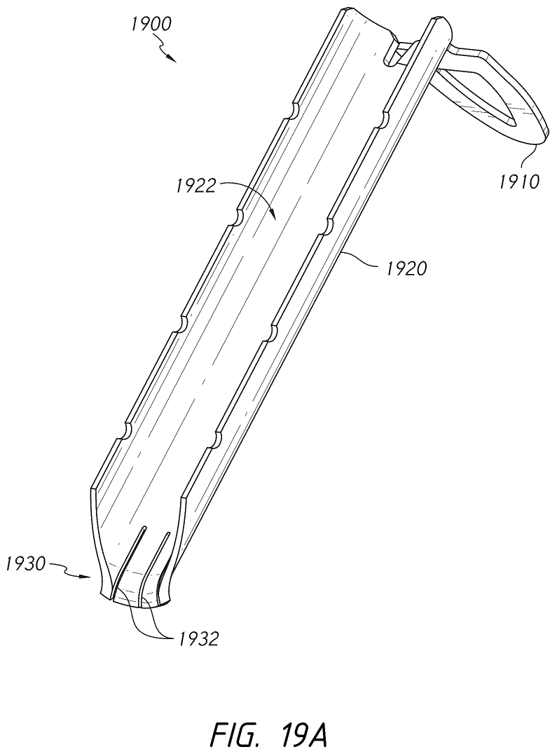

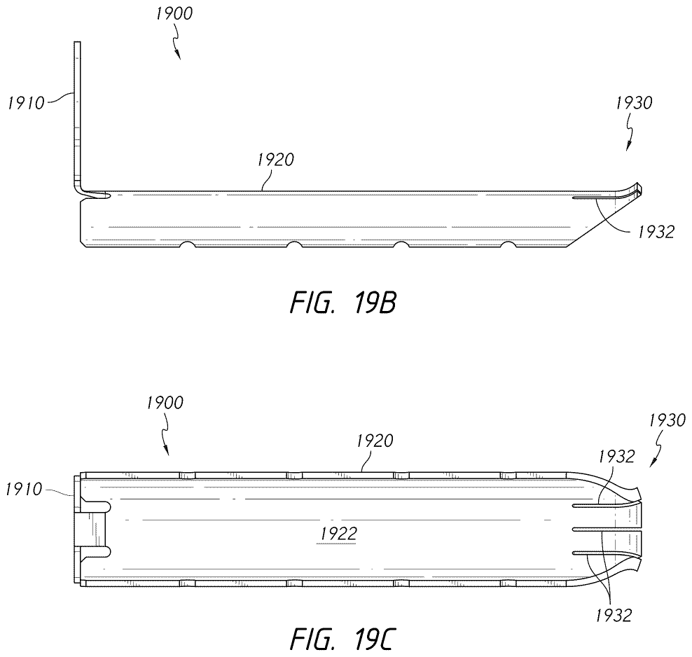

FIGS. 19A-19C are isometric, side, and front views, respectively, of a cannula in accordance with embodiments of the disclosure. Referring to FIGS. 19A-19C together, the cannula 1900 can include a cannula body 1920 and a flange 1910 extending from a proximal end of the cannula body 1920 substantially perpendicular to the cannula body 1920. The cannula body 1920 can have an elongate form extending along an axis and defining an open channel 1922. Thus, the cannula 1900 can be a split cannula. The cannula body 1922 can include a distal end portion 1930 having one or more slots 1932 extending from the distal tip of and into the cannula body 1922 (three slots 1932 are shown in the illustrated embodiment). It is appreciated that other embodiments can include a different number and/or different lengths of the slots 1932. Moreover, as best seen in FIG. 19B, the distal end portion 1930 can be flared in a direction away from the open channel 1922 (e.g., in the same direction as the flange 1910).

In operation, the cannula 1900 can be used to guide insertion of a surgical instrument, a spinal implant, and/or the like into a subject (e.g., via an incision). In particular, the one or more slots 1932 can define tissue-manipulators and/or cantilevered portions of the distal end portion 1930. For example, the cantilevered portions can act as fingers, grippers, claws, and/or the like to hold tissue. The number and configuration of the cantilevered portions can be selected based on the procedure. The flared geometry of the distal end portion 1930 can further facilitate holding tissue. For example, a user (e.g., a surgeon) can manipulate the cannula 1900 such that the rear surface of the distal end portion 1930 presses against and thereby holds tissue away from a workspace in the subject while, e.g., an endoscope positioned in the open channel 1922 is used to visualize the cleared workspace.

In some embodiments, the distal end portion 1930 can be actuatable. For example, the distal end portion 1930 can be rotated about one or more axes, can flex (e.g., between arched and flat configurations), and/or the like. The distal end portion 1930 can be actuatable via a knob that can be rotated and/or moved, pull-wires, shape memory material phase transitioning, and/or the like. The cantilevered portions may be actuatable together (e.g., using a single knob) or independently (e.g., using dedicated knobs).

It is appreciated that the distal end portion 1930 of the cannula 1900, and various embodiments thereof, whether actuatable or not, can be included in other cannulas (and/or other instruments). For example, the flared tips, the slots 1932, and/or the cantilevered portions described above with reference to FIGS. 19A-19C can be included in various open cannulas and closed cannulas, including those described in this application and in the patent references incorporated by reference herein in this application.

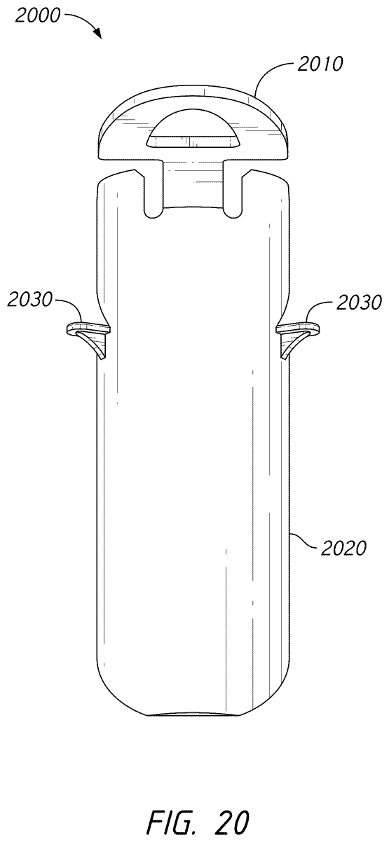

FIG. 20 is a rear view of a cannula 2000 configured in accordance with embodiments of the present technology. The cannula 2000 can include a cannula body 2020 and a flange 2010 extending from a proximal end of the cannula body 2020 substantially perpendicular to the cannula body 2020. The cannula body 2020 can have an elongate form extending along an axis and defining an open channel (obscured from view). Thus, the cannula 2000 can be a split cannula. The cannula body 2020 can include one or more anchors 2030 (e.g., protrusions, flanges, wings, tissue retention tabs) extending from either side of the cannula body 2020. In the illustrated embodiment, the two anchors 2030 extend from either side of an upper half of the cannula body 2020, generally laterally outward along a direction substantially perpendicular to the longitudinal axis of the cannula body 2020. It is appreciated that other embodiments can include a different number, position (e.g., along the length of the cannula body 2020), angle (e.g., relative to the longitudinal axis of the cannula body 2020), and/or length of the anchors 2030. The arrangement of the one or more anchors 2030 can be symmetrical or asymmetrical.

In some embodiments, the one or more anchors 2030 are integrally formed with the cannula body 2020 and are shaped to extend away therefrom, such as via cutting through and bending one or more portions of the cannula body 2020, molding, and/or the like. In some embodiments, the one or more anchors 2030 are coupled to the cannula body 2020, such as via soldering, welding, fasteners, adhesives, and/or the like. In some embodiments, the one or more anchors 2030 are actuatable. For example, the one or more anchors 2030 can be composed of a shape-memory material such that the one or more anchors 2030 can transition between a collapsed state (e.g., extending generally parallel to the longitudinal axis of the cannula body 2020) and a deployed state (e.g., extending generally perpendicular to the longitudinal axis of the cannula body 2020, as illustrated in FIG. 20). The one or more anchors 2030 can be blunt enough to avoid cutting or otherwise damaging tissue.

In operation, the cannula 2000 can be used to guide insertion of a surgical instrument, a spinal implant, and/or the like into a subject (e.g., via an incision). In particular, the one or more anchors 2030 can extend through or press against adjacent tissue and thereby anchor or stabilize a position of the cannula 2000. For example, the cannula 2000 can be inserted into an incision in the patient, then rotated by 90 degrees about the longitudinal axis of the cannula 2000 such that the one or more anchors 2030 engage soft tissue and thereby resist pull-out. In another example, the one or more anchors 2030 may be in a collapsed state during delivery to facilitate insertion into the patient, then the body temperature of the patient may transition the one or more anchors 2030 to the deployed state.

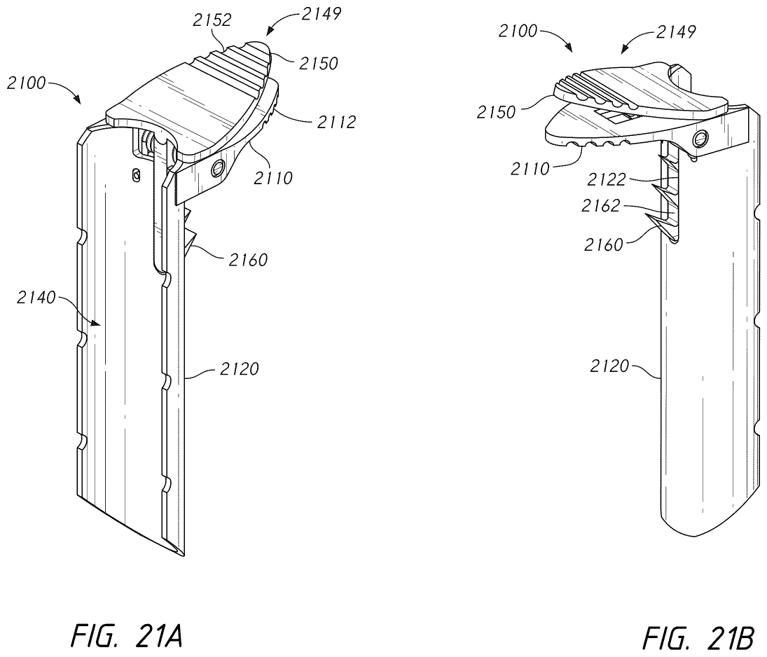

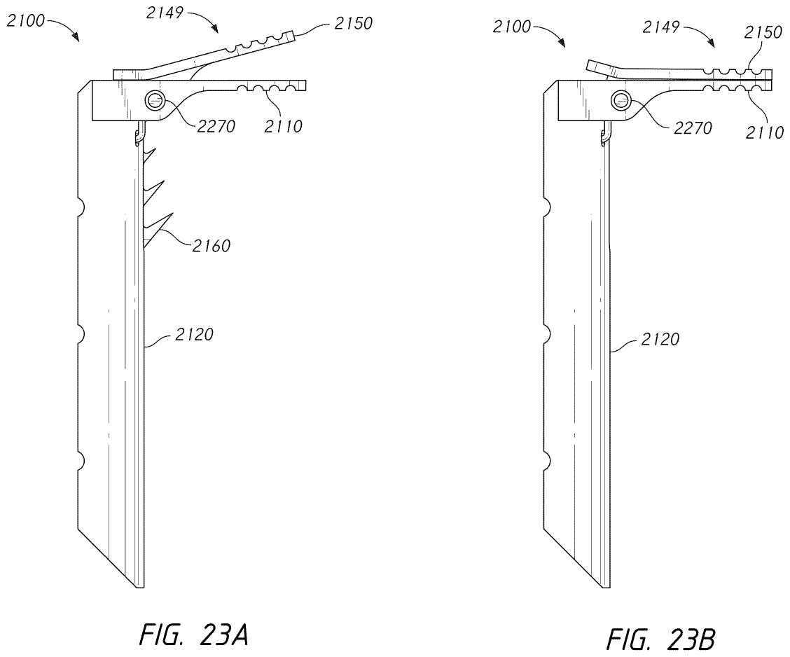

FIGS. 21A and 21B are front isometric and rear isometric views, respectively, of a split cannula assembly 2100 configured in accordance with embodiments of the present technology. Referring to FIGS. 21A and 21B together, the split cannula assembly 2100 can include a flange 2110, a cannula body 2120, and a deployable retention assembly 2149. The retention assembly 2149 can be moved between a deployed retention configuration (illustrated in FIGS. 21A and 21B) for engaging tissue and an undeployed configuration for repositioning or removal. The retention assembly 2149 can includes a lever 2150 (or other actuator) and one or more retention features 2160. The lever 2150 can be pivotally coupled to the cannula body 2120 and operated to move the one or more retention features 2160 between a deployed position and an undeployed position. Example operation of the retention assembly 2149 is discussed in connection with FIGS. 23A and 23B.

Referring to FIGS. 21A and 21B, the flange 2110 can extend from a proximal end of the cannula body 2120 substantially perpendicular to the cannula body 2120. The lower surface of the flange 2110 can include one or more grooves 2112 (FIG. 21A) that can improve grip. The cannula body 2120 can have an elongate form extending along an axis and defining an open channel 2140.

The lever 2150 can be positioned generally above the flange 2110. The upper surface of the lever 2150 can include one or more grooves 2152 (FIG. 21A) that can improve grip. The retention features 2160 can be barbs, hooks, and/or other protrusions having shapes suitable for gripping tissue. As illustrated in FIG. 21B, the retention features 2160 can be formed on and extend rearwardly from an elongate bar 2162, and the cannula body 2120 can include an elongate opening 2122 shaped and sized to receive the elongate bar 2162 therein. Thus, the elongate bar 2162 can extend partially along the length of the cannula body 2120 and be flush therewith.

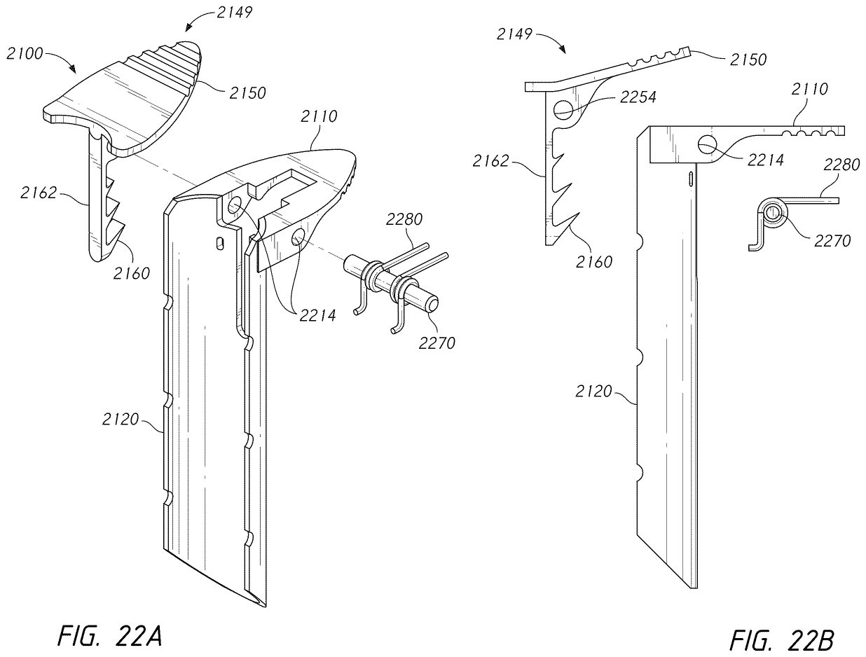

FIGS. 22A and 22B are partially exploded isometric and partially exploded side views, respectively, of the split cannula assembly 2100. Referring to FIGS. 22A and 22B together, the flange 2110 and the cannula body 2120 can be coupled together or integrally formed, and likewise, the lever 2150 and the elongate bar 2160 can be coupled together or integrally formed. Also, the split cannula assembly 2100 can further include a pivot rod 2270 and a biasing member 2280 (e.g., a torsion spring) coupled to or adjacent to the pivot rod 2270. In particular, the flange 2110 can include one or more first pivot openings 2214, the retention assembly 2149 can include one or more second pivot openings 2254, and upon assembly, the pivot rod 2270 can extend through each of the first pivot openings 2214 and the second pivot openings 2254. In some embodiments, the flange 2110 can be detachable from the cannula body 2120 to facilitate assembly.

FIGS. 23A and 23B illustrate operation of the split cannula assembly 2100. Specifically, FIG. 23A illustrates the split cannula assembly 2100 in a default or deployed state and FIG. 23B illustrates the split cannula assembly 2100 in an actuated or undeployed state. To switch the split cannula assembly 2100 from the default state to the actuated state, a physician or other user can operate the retention assembly 2149 by moving (e.g., by pressing down on the lever 2150, squeezing the lever 2150 and flange 2110) the lever 2150 toward the flange 2110 until the lever 2150 contacts and/or is substantially parallel to the flange 2110. Because the elongate bar 2162 (FIG. 22A) is fixedly coupled to the lever 2150, the elongate bar 2162 can rotate into the open channel 2140 (FIG. 21A). Consequently, the retention features 2160 can extend beyond the rear surface of the cannula body 2120 when in the default state (FIG. 23A), and can be positioned fully or substantially inside the open channel 2140 (e.g., retracted) when in the actuated state (FIG. 23B). The biasing member 2280 (FIGS. 22A and 22B) can bias the lever 2150 away from the flange 2110 such that when the user lets go of the lever 2150, the biasing member 2280 can return the split cannula assembly 2100 to the default state. In some embodiments, the lever 2150 can be replaced or supplemented with another actuation mechanism.

In operation, the physician can hold the split cannula assembly 2100 in the actuated state (FIG. 23B) by clamping on the lever 2150 and the flange 2110 (e.g., using their thumb and index finger). Because the retention features 2160 are retracted in the actuated state, the split cannula assembly 2100 can be more easily inserted into a port in the patient. When the split cannula assembly 2100 has been inserted to the desired depth, the physician can release the lever 2150 and allow the biasing member 2280 to return the split cannula assembly 2100 to the default state (FIG. 23A). Upon doing so, the retention features 2160 can press into adjacent patient tissue and provide grip to secure the position and orientation of the split cannula assembly 2100. To withdraw the split cannula assembly 2100 from the patient, the physician can press down on the lever 2150 again to switch to the actuated state (FIG. 23B) with the smaller profile for easier withdrawal.

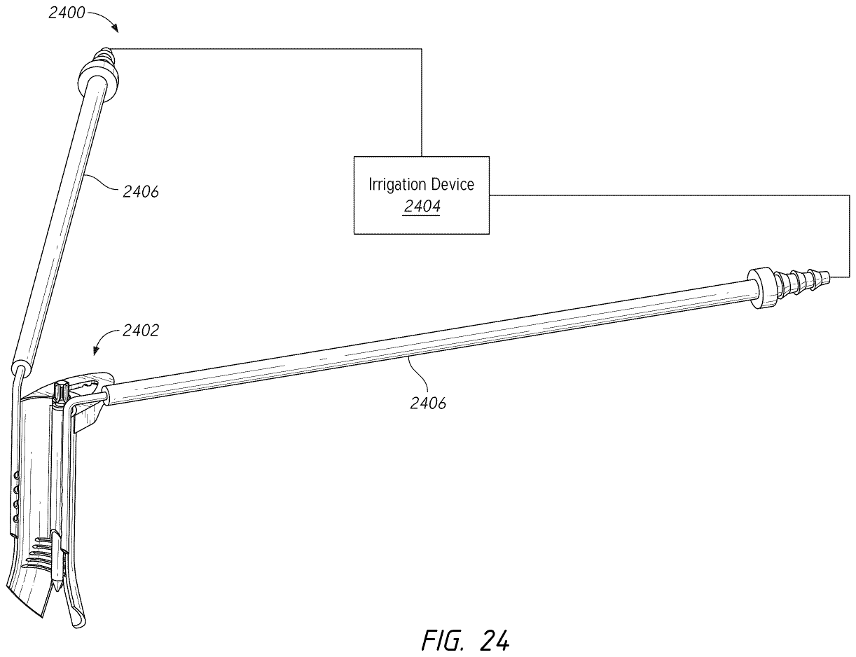

FIG. 24 is an isometric view of a cannula-irrigation system 2400 configured in accordance with embodiments of the present technology. The cannula-irrigation system 2400 can include a split cannula assembly 2402, an irrigation device 2404, and one or more fluid conduits or pipes 2406 coupled therebetween. The irrigation device 2404 can include one or more reservoirs, pumps, fluid systems, etc. and can provide and/or receive irrigation fluid (e.g., saline) using, e.g., a pump or suction device thereof to/from the fluid pipes 2406. For example, in the illustrated embodiment with the pair of fluid pipes 2406, the irrigation device 2404 may (i) provide irrigation fluid to both fluid pipes 2406, (ii) provide irrigation fluid to one fluid pipe 2406 and suction irrigation fluid from the other fluid pipe 2406, or (iii) suction irrigation fluid from both fluid pipes 2406. As discussed in greater detail herein, the split cannula assembly 2402 can include features for the transfer of irrigation fluid.

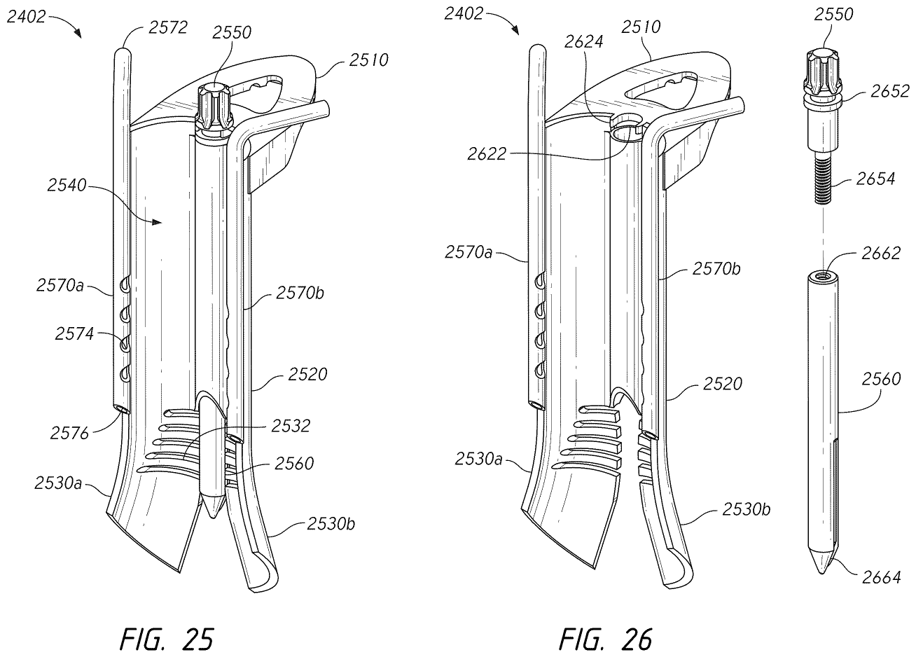

FIG. 25 is an isometric view of the split cannula assembly 2402. FIG. 26 is a partially exploded isometric view of the split cannula assembly 2402. Referring to FIGS. 25 and 26 together, the split cannula assembly 2402 can include a flange 2510, a cannula body 2520, a knob 2550, and a spreader 2560. The flange 2510 can extend from a proximal end of the cannula body 2520 substantially perpendicular to the cannula body 2520. In some embodiments, the lower surface of the flange 2510 includes one or more grooves (e.g., the grooves 2112 of FIG. 21A) that can improve grip. The cannula body 2520 can have an elongate form extending along an axis and defining an open channel 2540.

The cannula body 2520 can include a first distal end portion 2530a and a second distal end portion 2530b that can be spread apart, and one or more cutouts or slots 2532 to facilitate the associated mechanical deformation. The cannula body 2520 can also include an elongate opening 2622 (e.g., tube) shaped and sized to receive the knob 2550 and the spreader 2560. The knob 2550 can include a neck portion 2652 and a threaded portion 2654. When the knob 2550 is partially disposed in the elongate opening 2622, the neck portion 2652 can be positioned between the rim of the elongate opening 2622 and a protrusion 2624 of the flange 2510, thereby constraining vertical movement of the knob 2550. The flange 2510 may be detachable from the cannula body 2520 to facilitate assembly. The spreader 2560 can include a threaded opening 2662 that can threadedly receive and engage the threaded portion 2654 of the knob 2550, and a tapered distal end 2664. When the spreader 2560 is partially disposed in the elongate opening 2622, the tapered distal end 2664 can extend beyond the elongate opening 2622 and be positioned generally between the first distal end portion 2530a and the second distal end portion 2530b. The knob 2550 and the spreader 2560 can be operated in a similar manner as the knob 1270 and the disc-shaped portion 1280 of FIGS. 13A-16B to spread apart the first distal end portion 2530a and the second distal end portion 2530b, so a detailed discussion of their operation is omitted. Spreading and flaring the first distal end portion 2530a and the second distal end portion 2530b can help secure the split cannula assembly 2402 in position and improve retention thereat by pressing into adjacent tissue. Also, the spreading of the first distal end portion 2530a and the second distal end portion 2530b can provide additional soft tissue retraction.

The cannula body 2520 can further include one or more irrigation pipes (individually labeled 2570a and 2570b, collectively referred to as “the irrigation pipes 2570”). In the illustrated embodiment, two irrigation pipes 2570 are positioned on the two side edges of the cannula body 2520. Each irrigation pipe 2570 can include a first end opening 2572, one or more lateral openings 2574, and a second end opening 2576. The first end openings 2572 can be positioned adjacent to the flange 2510 and remain outside of the patient, and can be fluidically coupled to, e.g., the fluid pipes 2406 of FIG. 24 to be in communication with the irrigation device 2404. While the portions of the irrigation pipes 2570 near the first end openings 2572 are bent by 90° in the illustrated embodiment, the irrigation pipes 2570 can be straight or have other shapes in other embodiments. The second end openings 2576 can be positioned adjacent to the first distal end portion 2530a and the second distal end portion 2530b, and can remain in fluid communication with a working site inside the patient during operation.

The lateral openings 2574 can be spaced apart from one another along a length of the respective irrigation pipe 2570, and can generally face the open channel 2540. The number of lateral openings 2574 in each irrigation pipe 2570 can be at least one, two, three, four, five, or more. While in the illustrated embodiment, the lateral openings 2574 are formed in concave cutouts in the irrigation pipes 2570, in other embodiments, the lateral openings 2574 can be formed on the convex surfaces of the irrigation pipes 2570.

In operation, the physician or other user can utilize the irrigation device 2404 (FIG. 24) and the irrigation pipes 2570 of the split cannula assembly 2402 to provide irrigation of the working site. For example, if a particular irrigation pipe 2570 is used to provide delivery of irrigation fluid to the working site, the first end opening 2572 can serve as an inlet that receives the irrigation fluid, and the lateral openings 2574 and the second end opening 2576 can serve as outlets that release the irrigation fluid to the working site. Conversely, if a particular irrigation pipe 2570 is used to provide suction of irrigation fluid from the working site, the lateral openings 2574 and the second end opening 2576 can serve as inlets that receive the irrigation fluid, and the first end opening 2572 can serve as an outlet that releases the irrigation fluid back to the irrigation device 2404, which may be operably connected to a drain. Providing irrigation while the split cannula assembly 2402 is positioned in the patient, and thus during operation, can improve real-time visualization of the surgical site.

### IV. EXAMPLES

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner.
1. A split cannula assembly, comprising:
   a split cannula including one or more openings; and
   a retention device movably coupled to the split cannula, wherein the retention device includes one or more retention features and is movable relative the split cannula between an anchoring position for anchoring the split cannula to a patient and a non-anchoring position for moving the split cannula relative to the patient, wherein the one or more retention features extend through corresponding ones of the one or more openings of the split cannula to engage tissue of the patient when the retention device is in the anchoring position, and wherein the one or more retention features move away from the tissue when the retention device is moved toward the non-anchoring position.
2. The split cannula assembly of example 1, wherein the one or more retention features protrude from the split cannula when the retention device is in the anchoring position and move to a retracted position relative to the split cannula when the retention device is moved to the non-anchoring position.
3. The split cannula assembly of example 1 or example 2, wherein the split cannula and the retention device are translationally locked together when the retention device is in the anchoring position.
4. The split cannula assembly of any of examples 1-3, wherein each of the one or more retention features includes an anchor, a hook, a barb, or a flange.
5. The split cannula assembly of any of examples 1-4, wherein the split cannula includes an elongate split shaft, wherein the one or more retention features include at least two retention features, and wherein the one or more openings include at least two openings aligned along a length of the elongate split shaft.
6. The split cannula assembly of any of examples 1-5, wherein the split cannula includes a plurality of tubular holders configured to hold the retention device, and wherein the one or more openings are gaps between adjacent ones of the plurality of tubular holders.
7. The split cannula assembly of any of examples 1-6, wherein the split cannula includes a first elongate split shaft having a first curvature, and wherein the retention device includes a second elongate split shaft having a second curvature corresponding to the first curvature.
8. The split cannula assembly of any of examples 1-7, wherein the split cannula includes a plurality of tubular portions defining corresponding ones of a plurality of channels extending therethrough, wherein the retention device includes an elongate rod extending through the plurality of channels, and wherein the elongate rod is rotatable within the plurality of channels to move the one or more retention features along respective openings.
9. The split cannula assembly of any of examples 1-8, wherein:
   the split cannula includes a plurality of left tubular portions defining corresponding ones of a plurality of left channels extending therethrough, and a plurality of right tubular portions defining corresponding ones of a plurality of right channels extending therethrough,
   the retention device is a first retention device including a first elongate rod extending through the plurality of left channels, wherein the first elongate rod is rotatable within the plurality of left channels, and
   the split cannula assembly further comprises a second retention device including a second elongate rod extending through the plurality of right channels, wherein the second elongate rod is rotatable within the plurality of right channels.
10. The split cannula assembly of any of examples 1-9, wherein the split cannula includes an elongate split shaft defining an open channel, wherein the retention device includes an elongate rod, wherein the one or more retention features are arranged along one side of the elongate rod, and wherein the retention device is rotatable relative to the split cannula between (i) a delivery state in which the retention features face the open channel and (ii) a retention state in which the retention features face away from the open channel.
11. The split cannula assembly of any of examples 1-10, wherein the one or more retention features include a shape memory material configured to transition from a martensitic phase to an austenitic phase in response to body heat from the patient.
12. The split cannula assembly of any of examples 1-11, wherein the one or more retention features are actuatable from an extended position to a retracted position.
13. The split cannula assembly of any of examples 1-12, wherein the retention device includes a knob usable to rotate the retention device relative to the split cannula.
14. The split cannula assembly of any of examples 1-13, wherein the split cannula has a first length, and wherein the retention device has a second length shorter than the first length.
15. The split cannula assembly of any of examples 1-14, wherein the split cannula has a first insertion stop flange, and wherein the retention device has a second insertion stop flange configured to extend over and contact the first insertion stop flange.
16. The split cannula assembly of any of examples 1-15, wherein the retention device includes at least one biasing member configured to bias the retention device toward the anchoring position.
17. The split cannula assembly of any of examples 1-16, wherein the retention device includes a lever rotatably coupled to the split cannula and connected to the one or more retention features such that rotation of the lever relative to the split cannula causes movement of the one or more retention features relative to the split cannula.
18. The split cannula assembly of any of examples 1-17, further comprising an irrigation conduit extending along a sidewall of the split cannula, wherein the irrigation conduit includes a plurality of lateral openings facing an open channel of the split cannula.
19. The split cannula assembly of example 18, wherein the irrigation conduit includes a distal end with an end opening such that irrigation fluid flowing past the plurality of lateral openings exits the irrigation conduit via the end opening.
20 The split cannula assembly of example 18 or example 19, wherein the split cannula has an expandable distal end configured to move laterally outward past the irrigation conduit when driven to an expanded state.
21. A split cannula assembly, comprising:
   a split cannula including a first leg and a second leg; and
   an expander positioned at least partially between the first leg and the second leg, wherein the expander is operable to move at least one of the first leg or the second leg away from the other, thereby configuring the split cannula from a non-expanded state to an expanded state.
22. The split cannula assembly of example 21, wherein the split cannula has a material stiffness such that the first leg and the second leg are biased toward each other.
23. The split cannula assembly of example 21 or example 22, wherein the expander includes a wedge-shaped portion positioned to contact inner surfaces of the first leg and the second leg, wherein the wedge-shaped portion is configured to move axially to push against the inner surfaces and thereby spread the first leg and the second leg apart.
24. The split cannula assembly of any of examples 21-23, wherein the expander includes a first portion coupled to the split cannula and a second portion in threaded engagement with the first portion, wherein the first portion is rotatable to move the second portion between (i) a first position in which the second portion does not spread the first leg and the second leg away from the other and (ii) a second position in which the second portion spreads the first leg and the second leg away from the other.
25. The split cannula assembly of any of examples 21-24, wherein the first leg and the second leg are shaped to define a teardrop-shaped gap therebetween, wherein the expander includes:
   an elongate portion;
   a wedge-shaped portion coupled to a distal end portion of the elongate portion; and
   a disc-shaped portion coupled to a distal end portion of the wedge-shaped portion, wherein the wedge-shaped portion and the disc-shaped portion are movable in the teardrop-shaped gap to move the first leg and the second leg away from the other.
26. The split cannula assembly of any of examples 21-25, wherein the expander includes a knob usable to rotate the expander relative to the split cannula.
27. The split cannula assembly of any of examples 21-26, wherein the expander is operable to move both the first leg and the second leg simultaneously away from the other.
28. The split cannula assembly of any of examples 21-27, wherein the split cannula includes one or more cutouts configured to facilitate moving at least one of the first leg or the second leg away from the other.
29. The split cannula assembly of any of examples 21-28, wherein the expander has a wedge portion and a head that overlays adjacent edges of the split cannula, wherein the wedge portion pushes apart the first leg and the second leg when the head moves along a sidewall of the split cannula.
30 The split cannula assembly of any of examples 21-29, further comprising a retention device coupled to the split cannula, wherein the retention device includes at least one biasing member configured to bias the retention device toward an anchoring position such that anchors extend outwardly from the split cannula against tissue.
31. The split cannula assembly of example 30, wherein the retention device includes a lever rotatably coupled to the split cannula and one or more retention features coupled to the lever such that rotation of the lever relative to the split cannula causes movement of the one or more retention features relative to the split cannula.
32. The split cannula assembly of any of examples 21-31, further comprising an irrigation conduit extending along a sidewall of the split cannula and including a plurality of lateral openings facing toward an opening channel of the split cannula.
33 The split cannula assembly of example 32, wherein the irrigation conduit includes a distal end with an end opening such that irrigation fluid flowing past the plurality of lateral openings exits the irrigation conduit via the end opening.
34 The split cannula assembly of example 32 or example 33, wherein the split cannula has an expandable distal end configured to move laterally outward past the irrigation conduit when driven to an expanded state.

### V. CONCLUSION

It will be apparent to those having skill in the art that changes may be made to the details of the above-described embodiments without departing from the underlying principles of the present disclosure. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

To the extent any material incorporated herein by reference conflicts with the present disclosure, the present disclosure controls. Where the context permits, singular or plural terms may also include the plural or singular term, respectively. For example, throughout this disclosure, the singular terms “a,” “an,” and “the” include plural referents unless the context clearly indicates otherwise. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, a step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

Reference herein to “one embodiment,” “an embodiment,” “some embodiments,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10 (e.g., any and all subranges having a minimum value of equal to or greater than 1 and a maximum value of equal to or less than 10, such as 5.5 to 10).

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Features from various systems, methods, and instruments can be combined with features disclosed in U.S. application Ser. No. 15/793,950; U.S. application Ser. No. 17/902,685; U.S. Pat. Nos. 8,632,594; 9,308,099; 10,105,238; 10,201,431; 10,898,340; 11,464,648, PCT App. No. PCT/US20/49982; and PCT App. No. PCT/US22/21193; which are hereby incorporated by reference and made a part of this application. This application is related to U.S. Provisional Application No. 63/719,061, filed on Nov. 11, 2024, and U.S. Provisional Application No. 63/736,526, filed on Dec. 19, 2024, the disclosures of which are incorporated by reference herein in their entireties and made a part of this application. Variations of the split cannula assemblies are contemplated. For example, the disc-shaped portion 1280 of the expander 1250 (FIGS. 12A-16B) may be provided with different shapes and/or dimensions. In other examples, the various retention features described herein can have different geometries. Different materials may be combined in what is described herein as a single part.

Systems, components, and instruments disclosed herein can be disposable or reusable. For example, the split cannula assemblies can be disposable to prevent cross-contamination. As used herein, the term “disposable” when applied to a system or component (or combination of components), such as an instrument, a tool, or a distal tip or a head, is a broad term and generally means, without limitation, that the system or component in question is used a finite number of times and is then discarded. Some disposable components are used only once and are then discarded. In other embodiments, the components and instruments are non-disposable and can be used any number of times.

The disclosure set forth above is not to be interpreted as reflecting an intention that any claim or example requires more features than those expressly recited in that claim or example. Rather, as the preceding examples and the following claims reflect, inventive aspects lie in a combination of fewer than all features of any single foregoing disclosed embodiment. Thus, the preceding examples and the following claims are hereby expressly incorporated into the Detailed Description, with each claim standing on its own as a separate embodiment. This disclosure includes all permutations of the independent claims with their dependent claims.

What is claimed is:

1. A split cannula assembly, comprising:
a split cannula including one or more openings; and
a retention device movably coupled to the split cannula, wherein the retention device includes one or more retention features and is movable relative to the split cannula between an anchoring position for anchoring the split cannula to a patient and a non-anchoring position for moving the split cannula relative to the patient, wherein the one or more retention features extend through corresponding ones of the one or more openings of the split cannula to engage tissue of the patient when the retention device is in the anchoring position, and wherein the one or more retention features move away from the tissue when the retention device is moved toward the non-anchoring position.

2. The split cannula assembly of claim 1, wherein the one or more retention features protrude from the split cannula when the retention device is in the anchoring position and move to a retracted position relative to the split cannula when the retention device is moved to the non-anchoring position.

3. The split cannula assembly of claim 1, wherein the split cannula and the retention device are translationally locked together when the retention device is in the anchoring position.

4. The split cannula assembly of claim 1, wherein each of the one or more retention features includes an anchor, a hook, a barb, or a flange.

5. The split cannula assembly of claim 1, wherein the split cannula includes an elongate split shaft, wherein the one or more retention features include at least two retention features, and wherein the one or more openings include at least two openings aligned along a length of the elongate split shaft.

6. The split cannula assembly of claim 1, wherein the split cannula includes a plurality of tubular holders configured to hold the retention device, and wherein the one or more openings are gaps between adjacent ones of the plurality of tubular holders.

7. The split cannula assembly of claim 1, wherein the split cannula includes a first elongate split shaft having a first curvature, and wherein the retention device includes a second elongate split shaft having a second curvature corresponding to the first curvature.

8. The split cannula assembly of claim 1, wherein the split cannula includes a plurality of tubular portions defining corresponding ones of a plurality of channels extending therethrough, wherein the retention device includes an elongate rod extending through the plurality of channels, and wherein the elongate rod is rotatable within the plurality of channels to move the one or more retention features along respective openings.

9. The split cannula assembly of claim 1, wherein:
the split cannula includes a plurality of left tubular portions defining corresponding ones of a plurality of left channels extending therethrough, and a plurality of right tubular portions defining corresponding ones of a plurality of right channels extending therethrough;
the retention device is a first retention device including a first elongate rod extending through the plurality of left channels, wherein the first elongate rod is rotatable within the plurality of left channels; and
the split cannula assembly further comprises a second retention device including a second elongate rod extending through the plurality of right channels, wherein the second elongate rod is rotatable within the plurality of right channels.

10. The split cannula assembly of claim 1, wherein the split cannula includes an elongate split shaft defining an open channel, wherein the retention device includes an elongate rod, wherein the one or more retention features are arranged along one side of the elongate rod, and wherein the retention device is rotatable relative to the split cannula between (i) a delivery state in which the one or more retention features face the open channel and (ii) a retention state in which the one or more retention features face away from the open channel.

11. The split cannula assembly of claim 1, wherein the one or more retention features include a shape memory material configured to transition from a martensitic phase to an austenitic phase in response to body heat from the patient.

12. The split cannula assembly of claim 1, wherein the one or more retention features are actuatable from an extended position to a retracted position.

13. The split cannula assembly of claim 1, wherein the retention device includes a knob usable to rotate the retention device relative to the split cannula.

14. The split cannula assembly of claim 1, wherein the split cannula has a first length, and wherein the retention device has a second length shorter than the first length.

15. The split cannula assembly of claim 1, wherein the split cannula has a first insertion stop flange, and wherein the retention device has a second insertion stop flange configured to extend over and contact the first insertion stop flange.

16. The split cannula assembly of claim 1, wherein the retention device includes at least one biasing member configured to bias the retention device toward the anchoring position.

17. The split cannula assembly of claim 1, wherein the retention device includes a lever rotatably coupled to the split cannula and connected to the one or more retention features such that rotation of the lever relative to the split cannula causes movement of the one or more retention features relative to the split cannula.

18. The split cannula assembly of claim 1, further comprising an irrigation conduit extending along a sidewall of the split cannula, wherein the irrigation conduit includes a plurality of lateral openings facing an open channel of the split cannula.

19. The split cannula assembly of claim 18, wherein the irrigation conduit includes a distal end with an end opening such that irrigation fluid flowing past the plurality of lateral openings exits the irrigation conduit via the end opening.

20. The split cannula assembly of claim 18, wherein the split cannula has an expandable distal end configured to move laterally outward past the irrigation conduit when driven to an expanded state.

21. A split cannula assembly, comprising:
a split cannula including a first leg and a second leg; and
an expander positioned at least partially between the first leg and the second leg, wherein the expander is operable to move at least one of the first leg or the second leg away from the other, thereby configuring the split cannula from a non-expanded state to an expanded state.

22. The split cannula assembly of claim 21, wherein the split cannula has a material stiffness such that the first leg and the second leg are biased toward each other.

23. The split cannula assembly of claim 21, wherein the expander includes a wedge-shaped portion positioned to contact inner surfaces of the first leg and the second leg, and wherein the wedge-shaped portion is configured to move axially to push against the inner surfaces and thereby spread the first leg and the second leg apart.

24. The split cannula assembly of claim 21, wherein the expander includes a first portion coupled to the split cannula and a second portion in threaded engagement with the first portion, wherein the first portion is rotatable to move the second portion between (i) a first position in which the second portion does not spread the first leg and the second leg away from the other and (ii) a second position in which the second portion spreads the first leg and the second leg away from the other.

25. The split cannula assembly of claim 21, wherein the first leg and the second leg are shaped to define a teardrop-shaped gap therebetween, wherein the expander includes:
an elongate portion;
a wedge-shaped portion coupled to a distal end portion of the elongate portion; and
a disc-shaped portion coupled to a distal end portion of the wedge-shaped portion, wherein the wedge-shaped portion and the disc-shaped portion are movable in the teardrop-shaped gap to move the first leg and the second leg away from the other.

26. The split cannula assembly of claim 21, wherein the expander includes a knob usable to rotate the expander relative to the split cannula.

27. The split cannula assembly of claim 21, wherein the expander is operable to move both the first leg and the second leg simultaneously away from the other.

28. The split cannula assembly of claim 21, wherein the split cannula includes one or more cutouts configured to facilitate moving at least one of the first leg or the second leg away from the other.

29. The split cannula assembly of claim 21, wherein the expander has a wedge portion and a head that overlays adjacent edges of the split cannula, wherein the wedge portion pushes apart the first leg and the second leg when the head moves along a sidewall of the split cannula.

30. The split cannula assembly of claim 21, further comprising a retention device coupled to the split cannula, wherein the retention device includes at least one biasing member configured to bias the retention device toward an anchoring position such that anchors extend outwardly from the split cannula against tissue.

31. The split cannula assembly of claim 30, wherein the retention device includes a lever rotatably coupled to the split cannula and one or more retention features coupled to the lever such that rotation of the lever relative to the split cannula causes movement of the one or more retention features relative to the split cannula.

32. The split cannula assembly of claim 21, further comprising an irrigation conduit extending along a sidewall of the split cannula and including a plurality of lateral openings facing toward an opening channel of the split cannula.

33. The split cannula assembly of claim 32, wherein the irrigation conduit includes a distal end with an end opening such that irrigation fluid flowing past the plurality of lateral openings exits the irrigation conduit via the end opening.

34. The split cannula assembly of claim 32, wherein the split cannula has an expandable distal end configured to move laterally outward past the irrigation conduit when driven to an expanded state.
